SEQUENCE LISTING (56 sequences)

SEQ ID NO: 1 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 600 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGAIPVSLRGSGGSGGAEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 3 - protein, 630 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGEGTSTGSGAIPVSLRGSGGSGGAEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 609 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSEVQLVESGGGLVQPGGSLRLSCAASGFTFSSHWMTWFRQAPGKGLEWVAHIKEDGSEKYYEDSVEGRFTVSRDNAKNSVYLQMNSLRAEDTAVYYCARGGDGYSDSHFGVDVWGQGTTVTVSSGEGTSTGSGAIPVSLRGSGGSGGAEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 581 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGSGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 608 aa, synthetic construct
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGGGGSGGGSGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 366 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 8 - protein, 479 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSGEGTSTGSGGSGGSGGAEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 593 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 10 - protein, 623 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGEGTSTGSGGSGGSGGAEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 481 aa, synthetic construct
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 12 - protein, 459 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 610 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGGGSGGGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGAIPVSLRGSGGSGGAEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 14 - protein, 617 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSGEGTSTGSGAIPVSLRGSGGSGGAEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 576 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 590 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSGEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 360 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGGSGGGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSS

SEQ ID NO: 18 - protein, 600 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGAIPVSLRGSGGSGGAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 730 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSS

SEQ ID NO: 20 - protein, 630 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSAAAYPYDVPDYGSGEGTSTGSGAIPVSLRGSGGSGGAEPKSVDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 600 aa, synthetic construct; identical to SEQ ID NO: 18

SEQ ID NO: 22 - protein, 577 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 23 - protein, 591 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSGGGGSTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - skipped

SEQ ID NO: 25 - protein, 447 aa, synthetic construct
VQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLLSPGK

SEQ ID NO: 26 - protein, 673 aa, synthetic construct
VQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 360 aa, synthetic construct; identical to SEQ ID NO: 17

SEQ ID NO: 30 - protein, 592 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 360 aa, synthetic construct; identical to SEQ ID NO: 17

SEQ ID NO: 32 - protein, 588 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 33 - protein, 360 aa, synthetic construct; identical to SEQ ID NO: 17

SEQ ID NO: 34 - protein, 639 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 35 - protein, 360 aa, synthetic construct; identical to SEQ ID NO: 17

SEQ ID NO: 36 - protein, 589 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 360 aa, synthetic construct; identical to SEQ ID NO: 17

SEQ ID NO: 38 - protein, 592 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKYLSLSPG

SEQ ID NO: 39 - protein, 360 aa, synthetic construct; identical to SEQ ID NO: 17

SEQ ID NO: 40 - protein, 627 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPM

SEQ ID NO: 41 - protein, 360 aa, synthetic construct; identical to SEQ ID NO: 17

SEQ ID NO: 42 - protein, 608 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGEGTSTGSGGSGGSGGTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 43 - protein, 591 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTGGSEVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSSGGGGSGGGGSGGSGEPKSSDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 44 - protein, 506 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSTGGSQVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSSGGGGSGGGGSGSEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 4 aa, synthetic construct
TGGS

SEQ ID NO: 47 - protein, 5 aa, synthetic construct
GGGGS

SEQ ID NO: 48 - protein, 30 aa, synthetic construct
AAAYPYDVPDYGSGEGTSTGSGGSGGSGGA

SEQ ID NO: 49 - protein, 17 aa, synthetic construct
GEGTSTGSGGSGGSGGA

SEQ ID NO: 50 - protein, 117 aa, synthetic construct
EVQLVESGGGLVQPGGSLRLSCVASGDVHKINFLGWYRQAPGKEREKVAHISIGDQTDYADSAKGRFTISRDESKNMVYLQMNSLKPEDTAVYFCRAFSRIYPYDYWGQGTLVTVSS

SEQ ID NO: 51 - protein, 131 aa, synthetic construct
QVQLQESGGGLVQAGGSLRLSCAASGFTFDDYAIGWFRQAPGKEREGVSCIRVSDGSTYYADPVKGRFTISSDNAKNTVYLQMNSLKPEDAAVYYCAAGSLYTCVQSIVWPARPYYDMDYWGKGTQVTVSS

SEQ ID NO: 52 - protein, 217 aa, synthetic construct
APEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 127 aa, synthetic construct
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 54 - protein, 217 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 55 - protein, 7 aa, synthetic construct
EPKSCDK

SEQ ID NO: 56 - protein, 15 aa, synthetic construct
EPKSSDKTHTCPPCP

(19) United States
(12) Patent Application Publication
(10) Pub. No.: US 2025/0346681 A1
(43) Pub. Date: Nov. 13, 2025

(54) T CELL BINDING PROTEINS

(71) Applicant: AstraZeneca AB, Sodertalje (SE)

(72) Inventors: Mark COBBOLD, Wilmington, DE (US); Corinne CAYATTE, Gaithersburg, MD (US); Jonathan Christopher Joel SEAMAN, Cambridge (GB)

(21) Appl. No.: 18/854,215

(22) PCT Filed: Apr. 6, 2023
(86) PCT No.: PCT/EP2023/059278

§ 371 Date: Oct. 4, 2024

Related U.S. Application Data
(60) Provisional application No. 63/329,583, filed on Apr. 11, 2022.

(51) Int. Cl. C07K 16/28; A61K 39/00; A61P 35/00

(52) U.S. Cl. CPC C07K 16/2887; A61P 35/00; C07K 16/2809; C07K 16/2815; C07K 16/2878; A61K 2039/505; C07K 2317/31; C07K 2317/52; C07K 2317/92; C07K 2319/50

(57) ABSTRACT

The disclosure generally relates to binding proteins that comprise antigen binding sites, a T cell receptor binding site, and a T cell co-stimulatory molecule binding site. The disclosure also provides compositions comprising such binding proteins and nucleic acid molecules encoding such binding proteins. The disclosure further relates to methods of treating a disorder or condition using such binding proteins.

### CROSS-REFERENCE TO RELATED APPLICATIONS

This application claims priority to U.S. Provisional Application No. 63/329,583, filed Apr. 11, 2022, the contents of which are hereby incorporated by reference herein.

### FIELD OF THE DISCLOSURE

The disclosure generally relates to binding proteins that comprise antigen binding sites, a T cell receptor binding site, and a T cell co-stimulatory molecule binding site. The disclosure also provides compositions comprising such binding proteins and nucleic acid molecules encoding such binding proteins. The disclosure further relates to methods of treating a disorder or condition using such binding proteins.

### SEQUENCE LISTING STATEMENT

A computer readable form of the Sequence Listing is filed with this application by electronic submission and is incorporated into this application by reference in its entirety. The Sequence Listing is contained in the file created on Mar. 14, 2023 having the file name “21-0750-WO.xml” and is 71,648 bytes in size.

### BACKGROUND OF THE DISCLOSURE

Recruitment of T cell cytotoxic activity to destroy tumor cells is a worthwhile but complicated treatment strategy for cancer. The development of CD3-based bispecific T cell engagers (TCEs) as cancer therapeutics has been ongoing for the past 30 years. TCEs simultaneously bind a tumor associated antigen (TAA) and cluster of differentiation 3 (CD3) on a T cell to form a T cell receptor (TCR)-independent artificial immune synapse, circumventing human leukocyte antigen (HLA) restriction, and inducing T cell activation and cytolysis of the tumor cell.

The first generation of TCEs were simple bispecific T cell engagers (BiTEs), composed of two tandem single-chain variable fragments (scFvs) including a strong CD3-binding arm and a TAA binding domain. To date, there is only one Food and Drug Administration-approved BiTE, blinatumomab, which targets CD3 (using the Orthoclone OKT3 antibody) and cluster of differentiation 19 (CD19). The strong in vitro cytolytic activity observed during the development of BiTEs created excitement around their potential use for treating cancer. However, the unanticipated high cytokine release syndrome (CRS) observed in the clinic somewhat tempered that excitement. (Teachey et al., “Cytokine release syndrome after blinatumomab treatment related to abnormal macrophage activation and ameliorated with cytokine-directed therapy,” Blood 121: 5154-57 (2013)). Another observed disadvantage of BiTE formats is that they exhibit very short half-life and have poor manufacturability (Ellerman, “Bispecific T cell engagers: Towards understanding variables influencing the in vitro potency and tumor selectivity and their modulation to enhance their efficacy and safety,” Methods 154: 102-17 (2019)).

The second generation of TCEs include a fragment crystallizable (Fc) domain which can be modified to confer half-life extension and mutations to eliminate Fc receptor (FcR) binding, and present improved manufacturability (Vafa et al., “Perspective: Designing T cell Engagers With Better Therapeutic Windows,” Front Oncol. 10: 446 (2020)). Nevertheless, those molecules still include high affinity-CD3 binding domains, linking to induction of neurotoxicity and CRS in the clinic. More recent efforts have been focused on developing CD3 binding domains with reduced affinity with the hope to maintain potent T cell activation while significantly reducing associated cytokine release (Trinklein et al., “Efficient tumor killing and minimal cytokine release with novel T cell agonist bispecific antibodies,” MAbs 11: 639-52 (2019)).

Importantly, both CD3-based BiTEs and novel immunoglobulin G (IgG)-format TCEs bind and activate both cluster of differentiation 4 (CD4) and cluster of differentiation 8 (CD8) T cells, potentially engaging unfavorable T cells such as regulatory T cells (Treg), which have been shown to potentially decrease the cytolytic activity of CD8 T cells (Duell et al., “Frequency of regulatory T cells determines the outcome of the T cell-engaging antibody blinatumomab in patients with B-precursor ALL,” Leukemia 31: 2181-90 (2017)).

While T cell engager molecules offer promise, the therapeutic approach has faced challenges to date. There is a need in the art for improved T-cell binding proteins with increased activity and reduced off-target effects.

### SUMMARY OF THE DISCLOSURE

The disclosure provides a binding protein comprising four polypeptide chains that form two tumor-associated antigen binding sites, a T cell receptor binding site, and a T cell co-stimulatory molecule binding site, wherein the first and second polypeptide chains have a structure represented by the formula: V_{L}-C_{L} and a third polypeptide chain has a structure represented by the formula: V_{H1}-C_{H1}-V_{H2}-Fc and a fourth polypeptide chain has a structure represented by the formula: V_{H1}-C_{H1}-V_{H3}-Fc wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; V_{H3} is a heavy chain variable domain that specifically binds a T cell co-stimulatory molecule; and Fc is C_{H2} and C_{H3} immunoglobulin heavy chain constant domains.

Also provided herein is a binding protein comprising four polypeptide chains that form two tumor-associated antigen binding sites, a T cell receptor binding site, and a T cell co-stimulatory molecule binding site, wherein two polypeptide chains have a structure represented by the formula: V_{L}-C_{L} and two polypeptide chains have a structure represented by the formula: II₁-V_{H1}-C_{H1}-V_{H2}-Fc-II₂; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; Fc is C_{H2} and C_{H3} immunoglobulin heavy chain constant domains; II₁ and II₂ are each independently a heavy chain variable domain that specifically binds a T cell co-stimulatory molecule or are absent; wherein at least one of II₁ and II₂ is a heavy chain variable domain that specifically binds a T cell co-stimulatory molecule.

Also provided herein is a binding protein comprising four polypeptide chains that form two tumor-associated antigen binding sites, a T cell receptor binding site, and a T cell co-stimulatory molecule binding site, wherein two polypeptide chains have a structure represented by the formula: II₁-V_{L}-C_{L}-II₂; and two polypeptide chains have a structure represented by the formula: V_{H1}-C_{H1}-V_{H2}-Fc; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; Fc is C_{H2} and C_{H3} immunoglobulin heavy chain constant domains; II₁ and II₂ are each independently a heavy chain variable domain that specifically binds a T cell co-stimulatory molecule or are absent; wherein at least one of II₁ and II₂ is a heavy chain variable domain that specifically binds a T cell co-stimulatory molecule.

Also provided herein is a binding protein comprising three polypeptide chains that form two tumor-associated antigen binding sites, a T cell receptor binding site, and a T cell co-stimulatory molecule binding site, wherein the first polypeptide chain has a structure represented by the formula: V_{L}-C_{L}; and a second polypeptide chain has a structure represented by the formula: V_{H1}-C_{H1}-V_{H2}-Fc; and a third polypeptide chain has a structure represented by the formula: V_{H1}-V_{H3}-Fc; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; V_{H3} is a heavy chain variable domain that specifically binds T cell co-stimulatory molecule; and Fc is C_{H2} and C_{H3} immunoglobulin heavy chain constant domains.

Also provided herein is a binding protein comprising four polypeptide chains that form two tumor-associated antigen binding sites, a T cell receptor binding site, and a T cell co-stimulatory molecule binding site, wherein the polypeptide chains comprise (a) the amino acid of sequences of SEQ ID NO: 1, SEQ ID NO: 2, and SEQ ID NO: 3; (b) the amino acid of sequences of SEQ ID NO: 1, SEQ ID NO: 2, and SEQ ID NO: 4; (c) the amino acid of sequences of SEQ ID NO: 1, SEQ ID NO: 9, and SEQ ID NO: 10; (d) the amino acid of sequences of SEQ ID NO: 1, SEQ ID NO: 11, and SEQ ID NO: 12; (e) the amino acid of sequences of SEQ ID NO: 1, SEQ ID NO: 13, and SEQ ID NO: 14; (f) the amino acid of sequences of SEQ ID NO: 1 and SEQ ID NO: 19; (g) the amino acid of sequences of SEQ ID NO: 1, SEQ ID NO: 22, and SEQ ID NO: 23; (h) the amino acid of sequences of SEQ ID NO: 29 and SEQ ID NO: 30; (i) the amino acid of sequences of SEQ ID NO: 31 and SEQ ID NO: 32; (j) the amino acid of sequences of SEQ ID NO: 33 and SEQ ID NO: 34; (k) the amino acid of sequences of SEQ ID NO: 35 and SEQ ID NO: 36; (l) the amino acid of sequences of SEQ ID NO: 37 and SEQ ID NO: 38; (m) the amino acid of sequences of SEQ ID NO: 39 and SEQ ID NO: 40; (n) the amino acid of sequences of SEQ ID NO: 41 and SEQ ID NO: 42; or (o) the amino acid of sequences of SEQ ID NO: 45 and SEQ ID NO: 54.

Also provided herein is a binding protein comprising three polypeptide chains that form two tumor-associated antigen binding sites, a T cell receptor binding site, and a T cell co-stimulatory molecule binding site, wherein the polypeptide chains comprise the amino acid of sequences of (p) SEQ ID NO: 43, SEQ ID NO: 44, and SEQ ID NO: 45; or (q) the amino acid of sequences of SEQ ID NO: 1, SEQ ID NO: 11 and SEQ ID NO: 12.

### BRIEF DESCRIPTION OF THE DRAWINGS

The accompanying drawings are included to provide a further understanding of the methods and compositions of the disclosure and are incorporated in and constitute a part of this disclosure. The drawings illustrate one or more aspects of the disclosure and together with the description, serve to explain the principles and operation of the disclosure.

FIG. 1 illustrates a representative binding protein format of the present disclosure, including two binding sites to a tumor associated antigen (TAA), one binding site to T cell receptor (TCR) on T cells, and one binding site to a T cell costimulatory molecule. Upon cleavage by tumor specific protease at the site indicated, the Fc domain is released, and T cells are activated through TCR binding and activation of a costimulatory receptor.

FIGS. 2A-2B illustrate the structure of binding proteins that were evaluated in different in vitro assays. FIG. 2A illustrates the MCAZ 6.9 binding protein including protease cleavage sites.

FIG. 2B illustrates the MCAZ 6.10 binding protein including protease cleavage sites.

FIGS. 3A-3C show line graphs depicting assessment of MCAZ 6.9 and MCAZ 6.10 binding proteins in vitro cytolysis on CD20+ B cell lines. The EC50 of both MCAZ 6.9 and MCAZ 6.10 is shown for each cell line. FIG. 3A illustrates cytolysis assessment using Daudi cells and purified pan T cells. FIG. 3B illustrates cytolysis assessment using Ramos cells and purified pan T cells. FIG. 3C illustrates cytolysis assessment using Raji cells and PBMCs.

FIGS. 4A-4B show line graphs depicting percentage of surface CD25+ cells using Daudi cells as a means of analyzing CD4 and CD8 T cell activation profiles. FIG. 4A shows results with the MCAZ 6.9 binding protein. FIG. 4B shows results with the MCAZ 6.10 binding protein.

FIGS. 5A-5B show line graphs depicting percentage of surface CD25+ cells using Ramos cells. CD25+ assessment is used as a means of analyzing CD4 and CD8 T cell activation profiles. FIG. 5A shows results with the MCAZ 6.9 binding protein. FIG. 5B shows results with the MCAZ 6.10 binding protein.

FIG. 6 shows a line graph depicting percentage of surface CD25+ cells using Raji cells as a means of analyzing CD4 and CD8 T cell activation profiles.

FIGS. 7A-7C show in vitro cytolysis and T cell activation profiles induced by CD20 T cell MCAZ 88 binding protein. FIG. 7A shows a representative structure of the MCAZ 88 binding protein. FIG. 7B shows that MCAZ 88 induced cytolysis assessed on Raji cell line (CD20+, 85371 antigen per cell). FIG. 7C shows CD4 and CD8 T cell activation profiles assessed by measuring the % of surface CD25+ cells.

FIG. 8A is schematic representation of MCAZ 7.5 and MCAZ 89. FIGS. 8B-8C show bar graphs illustrating non-specific CD4 and CD8 T cell activation in solution as assessed by flow cytometry detecting CD25+ status. Binding proteins as shown from top to bottom in the legend are depicted from left to right along the x axis. FIG. 8B shows non-specific CD8 activation. FIG. 8C shows non-specific CD4 activation.

FIGS. 9A-9B show bar graphs illustrating non-specific CD8 and CD4 T cell activation in plate-adsorbed antibodies as assessed by flow cytometry detecting CD25+ surface expression. FIG. 9A shows non-specific CD8 activation. Binding proteins as shown from top to bottom in the legend are depicted from left to right along the x axis. FIG. 9B shows non-specific CD4 activation.

FIGS. 10A-10E show in vitro cytolysis and T cell activation profiles induced by uncleavable binding proteins. FIG. 10A shows the structure of the MCAZ 7.1 binding protein.

FIG. 10B shows the structure of the MCAZ 10.3 binding protein. FIG. 10C shows that cytolysis was assessed on CD20+ B cell lines Toledo, Oci-LY18, and SU-DHL5 (respectively expressing 12420, 20244, and 27152 CD20 antigen per cell). FIG. 10D shows MCAZ 7.1 cytolysis EC50 values for each cell line. FIG. 10E shows CD4 and CD8 T cell activation assessed as % CD25 expressing cells.

FIGS. 11A-11H show in vitro cytolysis, specific and non-specific T cell activation profiles induced by binding proteins with modified linker length. FIG. 11A shows the structure of the MCAZ 7.7 binding protein including an additional linker between the CD20 binding domain and the TCR binding domain. FIG. 11B shows that cytolysis was assessed on CD20+ Raji B cell line (expressing 85000 CD20 antigen per cell). FIG. 11C shows CD4 and CD8 T cell activation profiles assessed as % CD25 expressing cells. FIG. 11D shows binding proteins were plate-bound and 1.5e5 purified T cells were added, in the absence of tumor cells and associated proteases. Non-specific CD8 and CD4 T cell activation levels were assessed by flow cytometry as % CD69+/CD25+ surface expression. FIG. 11E shows the structure of the MCAZ 10.1 binding protein with a more rigid CD8 arm by removal of TGGS (SEQ ID NO: 46) linker and HA tag, in addition to the removal of cleavable linkers. FIG. 11F shows that cytolysis was assessed on OCI-Ly18 B cell line. B cell lines were CTV stained and incubated PBMCs at an E:T ratio of 5:1 for 3 days. % cytolysis was measured by flow cytometry. FIGS. 11G and 11H show CD8 and CD4 T cell activation profile were assessed as % CD25 expressing cells.

FIGS. 12A-12E show the evaluation of the impact of CD8 positioning on the binding protein. FIG. 12A shows the structure of the MCAZ 8.71 binding protein. FIG. 12B shows the structure of MCAZ 8.81 binding protein. FIG. 12C shows cytolysis was assessed on CD20+ B Raji cell line (expressing 85000 CD20 antigen per cell). FIG. 12D shows cytolysis EC50 values for each cell line. FIG. 12E shows CD4 and CD8 T cell activation profile assessed as % CD25 expressing cells.

FIGS. 13A-13E show evaluation of TCR VHH and CD8 VHH bispecific binding proteins. FIG. 13A shows the structure of the MCAZ 8.69 binding protein. FIG. 13B shows the structure of the MCAZ 8.70 binding protein. FIG. 13C shows cytolysis assessed on CD20+ B Raji cell line (expressing 85000 CD20 antigen per cell). FIG. 13D shows cytolysis EC50 values for each cell line. FIG. 13E shows CD4 and CD8 T cell activation profiles assessed as % CD25 expressing cells.

FIGS. 14A-14B show non-specific T cell activation assessment for various binding proteins. Different concentrations of binding proteins were plate-bound and 1.5e5 purified T cells were added, in the absence of tumor cells and associated proteases. After 48 h incubation, unspecific (FIG. 14A) CD8 and (FIG. 14B) CD4 T cell activation levels were assessed by flow cytometry as % CD69+/CD25+ surface expression.

FIG. 15A is a schematic of MCAZ 8.71 binding proteins with different Fc regions. FIG. 15B shows the cytolytic activity of the binding proteins at 72 h on OCI-Ly18 B cell line, with PBMCs at E:T ratio of 5:1. FIGS. 15C and 15D show CD8 and CD4 T cell activation profiles assessed by measuring the % of surface CD25+ T cells.

FIGS. 16A-16C show the cytolytic activity of a MCAZ 7.1 variant (TENG0093) with modified linkers. Specifically, the variant included similar linkers on the CD8 and TCR VHH arms as indicated on FIG. 16A. The variant had a different profile than a broad CD3×CD20 bivalent engager. FIG. 16A shows the structure of the modified MCAZ 7.1 with different linkers and the broad CD3×CD20 bivalent engager used as the comparator. FIG. 16B shows the cytolytic activity of the MCAZ 7.1 variant and CD3×CD20 bivalent engager on OCI-Ly18 B cell line. FIG. 16C shows the CD4 and CD8 T cell activation profiles assessed at 72 h as surface expression of CD25.

FIGS. 17A-17F show that MCAZ 7.1 exhibited strong binding to CD8 T cells and induced preferential association of CD20+ tumor cells with CD8 T cells compared to the broad CD3×CD20 bivalent engager. FIG. 17A shows MCAZ 7.1 binding profile on CD20+ tumor B cell line (OCI-Ly-18), on (FIG. 17B) purified CD4 and (FIG. 17C) CD8 T cells from PBMCs from healthy donors. FIG. 17D shows magnification of the CD3×CD20 bivalent engager binding to CD8 T cells. The % of CD8-B cell conjugates and CD4-T cell conjugate was assessed by flow cytometry after staining of CD4 and CD8 T cells after (FIG. 17E) incubation with MCAZ 7.1 and (FIG. 17F) the CD3×CD20 bivalent engager.

FIGS. 18A-18F show that MCAZ 7.1 selective engagement of CD8 T cells during cytolysis was associated with significantly lower cytokine release than broad CD3+ T cell engagement by the CD3×CD20 bivalent engager. The MCAZ 7.1 binding protein and the CD3×CD20 bivalent engager were incubated with OCI-Ly18 B cell line and PBMCs at an E:T ratio of 5:1 for 72 h. Supernatants were harvested and analyzed by multiplex assay to measure the concentrations of released pro-inflammatory cytokines: (FIG. 18A) TL-6, (FIG. 18B) TNF-α, (FIG. 18C) IL-10, (FIG. 18D) IFN-g, (FIG. 18E) IL-2, and (FIG. 18F) IL-17A.

FIGS. 19A-19B show that MCAZ 7.1 did not induce significant level of T cell activation and cytokine release compared to the CD3×CD20 bivalent engager. Various concentrations of the MCAZ 7.1 binding protein and the CD3×CD20 bivalent engager were plate-bound and 1.5e5 PBMCs from healthy donor was added and incubated for 48 h. FIG. 19A shows CD4 and CD8 non-specific T cell activation profiles assessed for CD25/CD69+ surface expression levels by flow cytometry. FIG. 19B shows multiplex assay measuring the concentrations of released pro-inflammatory cytokines for harvested supernatants.

FIGS. 20A-20B show that the MCAZ 7.1 variant (TENG0093) binding protein and the CD3×CD20 bivalent engager induced strong cytolytic activity in a 3D-spheroid model. GFP-expressing CD20+ B cell line (TMD8, 100 000 CD20/cell) were plated in low adherent plate to form 3D-spheroids for 72 h. Then, purified panT cells were added at a 15:1 E:T ratio and co-incubated with no engager, the MCAZ 7.1 variant binding protein, or the CD3×CD20 bivalent engager for 96 h. FIG. 20A shows a representative image of spheroids in the absence of binding protein, with the MCAZ 7.1 variant binding protein or with the CD3×CD20 bivalent engager. FIG. 20B shows the average % of GFP signal intensity relative to no engager control at various concentrations of T cell engagers after 96 hours incubation.

FIG. 21 shows PK assessment after a single dose of the MCAZ 7.1 binding protein. NSG mice humanized with panT cells were injected with 0.5 mg/kg of binding protein as indicated and systemic concentrations of binding proteins were measured after 1 h, 6 h, 24 h, 48 h, 72 h, and 168 h.

FIG. 22 shows in vivo efficacy of the MCAZ 7.1 binding protein in B cell lymphoma in a humanized NSG mouse model. NSG mice were engrafted with panT cells two days prior to the study start. 5e6 OCI-Ly18 CD20+ tumor cell lines were implanted s.c. at day 0 and animals were dosed i.p. with 1 mg/kg of MCAZ 7.1 binding protein or CD3×CD20 bivalent engager at day 2, followed by weekly dosing at the same concentration as indicated by the grey arrows under the X axis.

FIGS. 23A-23F show in vivo cytokine release assessment of the MCAZ 7.1 binding protein compared to the CD3×CD20 bivalent engager in a CRS mouse model. Mean+/−SD are plotted for (FIG. 23A) TL-6, (FIG. 23B) TNF-α, (FIG. 23C) IL-10, (FIG. 23D) IFN-g, (FIG. 23E) IL-2, and (FIG. 23F) IL-17A.

FIGS. 24A-24H show binding protein in vitro activity for MCAZ 7.1 and the MCAZ 7.1 variant on PBMCs from Non-Hodgkin lymphoma (NHL) donors. B cell killing was assessed on PBMCs from NHL (DLBCL) donors. The MCAZ 7.1 variant binding protein was spiked in the PBMCs at the indicated concentrations and 48 h later, % B cells was assessed by flow cytometry for each patient (FIGS. 24A, 24C, 24E and 24G). The % B cell cytolysis is presented at the different concentrations of binding proteins indicated for 3 different DLBCL donors (FIGS. 24B, 24D, 24F, 24H) CD4 and CD8 T cell activation profiles were assessed as % of CD25+ T cells.

FIGS. 25A-25B show a representative molecular format of EGFR TITAN and a broad CD3 engager that was used as a comparator.

FIGS. 26A-26B show the binding profiles of EGFR TITAN (MCAZ13.8) and broad CD3 comparator on NCIH196 (26A) and MDA-MB231 (26B).

FIGS. 27A-27B show in vitro cytotoxicity and T cell activation profiles for NCIH196-EGFR high. 27A shows equivalent cytolytic activities of EGFR TITAN and the broad CD3 engager used as a comparator on the NCIH196 tumor cell line. 27B shows strong biased CD8 T cell activation profile for EGFR TITAN (MCAZ13.8) as highlighted by the % CD25 surface expression on T cells.

FIGS. 28A-28E show that EGFR TITAN (MCAZ13.8) selective engagement of CD8 T cells during cytolysis is associated with significantly lower cytokine release than broad CD3+ T cell engagement by CD3×EGFR bivalent comparator.

FIGS. 29A-29B show in vitro cytotoxicity and T cell activation bias for MDA-MB-231-EGFR high. FIG. 29A shows equivalent cytolytic activities of EGFR TITAN and broad CD3 engager used as a comparator on MDA-MB-231 tumor cell line. FIG. 29B shows strong biased CD8 T cell activation profile for EGFR TITAN (MCAZ13.8) as highlighted by the % CD25 surface expression on T cells.

FIGS. 30A-30E show that EGFR TITAN (MCAZ13.8) selective engagement of CD8 T cells during cytolysis is associated with significantly lower cytokine release than broad CD3+ T cell engagement by CD3×EGFR bivalent comparator.

FIGS. 31A-31B show T cell activation. FIG. 31A shows that EGFR TITAN (MCAZ 13.8) binding protein does not activate T cells when plate-bound at different concentrations. In comparison the broad CD3 engager used as a comparator induces significant T cell activation at the highest concentrations used. FIG. 31B shows that the absence of T cell activation induced by plate-bound EGFR TITAN (MCAZ 13.8) is associated with absence of cytokine release.

FIGS. 32A-32C show TENG0093 potently depletes B cells in fully humanized NSG mice. NSG-SGM3 mice were humanized with human stem cells (CD34+ cells). Fourteen weeks later, animals were treated I.P. with 20 mg/kg of Fc block and 16 h later treated I.P. with 1 mg/kg of TENG0093 or CD3×CD20 bivalent engager. At day 6 post-engager treatment, B cell depletion efficacy was assessed by flow cytometry using anti-CD19 surface staining. Both CD20 T cell engagers induced potent and nearly complete B cell depletion efficacy in blood (32A), spleen (32B), and bone marrow (32C) compared to PBS treated groups (no treatment group).

### DETAILED DESCRIPTION OF THE DISCLOSURE

The disclosure generally relates to binding proteins that comprise antigen binding sites, a T cell receptor binding site, and a T cell co-stimulatory molecule binding site. The disclosure also provides compositions comprising such binding proteins and nucleic acid molecules encoding such binding proteins. The disclosure further relates to methods of treating a disorder or condition using such binding proteins.

The binding proteins disclosed herein preferentially bind to CD8+ T cells. As a result of the preferential binding to CD8+ T cells, the binding proteins avoid engagement with tumor promoting T cells such as Treg, Th2 and Th17 cells and avoid engagement with CD4+ T cells which produce the majority of cytokines that cause release syndrome (CRS). By reducing the fraction of T cells that are activated, the binding proteins disclosed herein reduce CRS. Additionally, the CD8+ T cells bound by the binding proteins disclosed herein induce a type of programmed cell death termed pyroptosis that is immunogenic. A pyroptotic cell is taken up by antigen presenting cells and may drive further tumor-specific T cell responses.

It is to be understood that the particular aspects of the disclosure are described herein are not limited to specific aspects presented and can vary. It also will be understood that the terminology used herein is for the purpose of describing particular aspects only and, unless specifically defined herein, is not intended to be limiting. Moreover, particular aspects disclosed herein can be combined with other aspects disclosed herein, as would be recognized by a skilled person, without limitation.

Unless otherwise indicated or otherwise evident from the context and understanding of one of ordinary skill in the art, values herein that are expressed as ranges can assume any specific value or sub-range within the stated ranges in different aspects of the disclosure, to the tenth of the unit of the lower limit of the range, unless the context clearly dictates otherwise.

Throughout this disclosure, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

The term “binding protein,” as used herein, refers to a non-naturally occurring (or recombinant) molecule which comprises multiple polypeptide chains that form at least one antigen binding site.

A “recombinant” molecule is one that has been prepared, expressed, created, or isolated by recombinant DNA technology means.

The term “antibody,” as used herein, refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The variable domain may be subjected to further protein engineering to humanize the framework regions if the antibody was derived from a non-human source. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

“Antigen-binding fragment thereof” refer to at least the minimal portion of an antibody which is capable of binding to a specified antigen which the antibody targets, e.g., at least some of the complementarity determining regions (CDRs) of the variable domain of a heavy chain (V_{H}) and the variable domain of a light chain (V_{L}) in the context of a typical antibody produced by a B cell. Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fvs, single-chain Fvs (scFvs), single-chain antibodies, disulfide-linked Fvs (sdFvs), fragments comprising either a V_{L} or V_{H} domain alone or in conjunction with a portion of the opposite domain (e.g., a whole V_{L} domain and a partial V_{H} domain with one, two, or three CDRs), and fragments produced by a Fab expression library. ScFv molecules are known in the art and are described, e.g., in U.S. Pat. No. 5,892,019.

The term “native Fc,” as used herein, refers to a molecule comprising the sequence of a non-antigen binding fragment resulting from digestion of an antibody or produced by other means, whether in monomeric or multimeric form, and can contain the hinge region. The original immunoglobulin source of the native Fc is preferably of human origin and can be any of the immunoglobulins. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc,” as used herein, is generic to the monomeric, dimeric, and multimeric forms.

The term “Fc variant,” as used herein, refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed or mutated to produce an Fc variant to alter certain residues that provide structural features or biological activity that are not required for the binding proteins of the disclosure. Thus, the term “Fc variant” comprises a molecule or sequence that lacks one or more native Fc sites or residues, or in which one or more Fc sites or residues has been modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “Fc,” as used herein, encompasses native Fc and Fc variants as defined above. As with Fc variants and native Fc molecules, the term “Fc” includes molecules in monomeric or multimeric form, whether digested from whole antibody or produced by other means.

Binding proteins encompassed by this disclosure can be of or be derived from any type (e.g., IgG, IgE, IgM, IgD, IgA, and IgY), class (e.g., IgG1, IgG2, IgG3, IgG4, IgA1, and IgA2), or subclass of immunoglobulin molecule.

The term “antigen” or “target antigen,” as used herein, refers to a molecule or a portion of a molecule that is capable of being recognized by and bound by the antigen binding portion of the binding proteins of the disclosure. The target antigen is capable of being used in an animal to produce antibodies capable of binding to an epitope of that antigen. A target antigen may have one or more epitopes. With respect to each target antigen recognized by the antigen binding portion of the binding protein, is capable of competing with an intact antibody that recognizes the target antigen.

The term “antigen binding site,” as used herein, refers to a site created on the surface of a binding protein of the disclosure where an antigen or an epitope on an antigen is bound.

The term “linker,” as used herein, refers to one or more amino acid residues inserted between domains of the binding protein of the disclosure. For example, a linker may be inserted between domains, at the sequence level. The precise location of a domain transition can be determined by locating peptide stretches that do not form secondary structural elements such as beta-sheets or alpha-helices as demonstrated by experimental data or as can be assumed by techniques of modeling or secondary structure prediction. Linkers may or may not be needed depending on the where the stop and start residues of protein fusions are chosen because often natural linkers are found between immunoglobulin domains.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA.

As used herein, the term “vector” can refer to a nucleic acid molecule as introduced into a host cell, thereby producing a transformed host cell. A vector can include nucleic acid sequences that permits it to replicate in a host cell, such as an origin of replication. A vector can also include one or more selectable marker gene and other genetic elements known in the art. Specific types of vector envisioned here can be associated with or incorporated into viruses to facilitate cell transformation.

As used herein, the terms “treat,” “treatment,” or “treatment of” refer to reducing disease pathology, reducing or eliminating disease symptoms, promoting increased survival rates, and/or reducing discomfort. For example, treating can refer to the ability of a therapy to reduce disease symptoms, signs, or causes when administered to a subject. Treating also refers to mitigating or decreasing at least one clinical symptom and/or inhibition or delay in the progression of the condition and/or prevention or delay of the onset of a disease or illness.

The terms “administration” or “administering,” as used herein, refer to providing, contacting, and/or delivering a binding protein by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a binding protein, is an amount sufficient to carry out a specifically stated or intended purpose, such as treating or treatment of cancer. An “effective amount” can be determined empirically in a routine manner in relation to the stated purpose.

The term “pharmaceutical composition,” as used herein, refer to a compound or composition capable of inducing a desired therapeutic effect when properly administered to a subject. In some aspects, the disclosure provides a pharmaceutical composition comprising a pharmaceutically acceptable carrier and a therapeutically effective amount of binding proteins of the disclosure. The terms “pharmaceutically acceptable carrier” or “physiologically acceptable carrier,” as used herein, refer to one or more formulation materials suitable for accomplishing or enhancing the delivery of one or more binding proteins of the disclosure.

In some aspects, the binding proteins disclosed herein may be formulated with a pharmaceutically acceptable carrier, excipient, or stabilizer, as pharmaceutical compositions. In certain aspects, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, for example, as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In some aspects provided herein is a binding protein comprising two tumor-associated antigen (TAA) binding sites. In some aspects, the tumor associated antigen (TAA) is cluster of differentiation 20 (CD20). CD20 is a transmembrane protein involved in Ca⁺⁺ channeling, B-cell activation, and proliferation. CD20 is a membrane-embedded surface molecule which plays a role in the development and differentiation of B-cells into plasma cells. In some aspects, the binding protein comprises a fragment of rituximab (see, e.g., U.S. Pat. No. 5,736,137).

In some aspects, provided herein is a binding protein comprising one T cell receptor (TCR) binding site. The TCR comprises a heterodimer including the highly variable alpha (α) and beta (β) chains. The multicomponent complex of the TCR comprises the CD3 co-receptor, which plays a significant role in activating T cells.

In some aspects provided herein is a binding protein comprising one T cell costimulatory molecule binding site. A co-stimulatory molecule comprises a co-stimulatory domain capable of potentiating or modulating the response of immune effector cells. Co-stimulatory domains can include sequences, for example, from one or more of CD3zeta (or CD3z), CD28, CD137 (4-1BB), OX-40, ICOS, CD27, GITR, CD2, IL-2Rβ and MyD88/CD40. In some aspects, the T cell costimulatory molecule is CD8. In some aspects, the T cell costimulatory molecule is CD137 (4-1BB).

Some aspects described herein provide a binding protein comprising four polypeptide chains that form two tumor-associated antigen binding sites, a T cell receptor binding site, and a T cell co-stimulatory molecule binding site, wherein the first and second polypeptide chains have a structure represented by the formula: V_{L}-C_{L}; and a third polypeptide chain has a structure represented by the formula: V_{H1}-C_{H1}-V_{H2}-Fc; and a fourth polypeptide chain has a structure represented by the formula: V_{H1}-C_{H1}-V_{H3}-Fc; wherein V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; V_{H3} is a heavy chain variable domain that specifically binds a T cell co-stimulatory molecule; and Fc is an immunoglobulin hinge region and CH₂ and CH₃ immunoglobulin heavy chain constant domains.

Some aspects described herein provide a binding protein comprising four polypeptide chains that form two tumor-associated antigen binding sites, a T cell receptor binding site, and a T cell co-stimulatory molecule binding site, wherein two polypeptide chains have a structure represented by the formula: V_{L}-C_{L} and two polypeptide chains have a structure represented by the formula: II₁-V_{H1}-C_{H1}-V_{H2}-Fc-II₂; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; Fc is an immunoglobulin hinge region and CH₂ and CH₃ immunoglobulin heavy chain constant domains; II₁ and II₂ are each independently a heavy chain variable domain that specifically binds a T cell co-stimulatory molecule or are absent; wherein at least one of II₁ and II₂ is a heavy chain variable domain that specifically binds a T cell co-stimulatory molecule.

Some aspects described herein provide a binding protein comprising four polypeptide chains that form two tumor-associated antigen binding sites, a T cell receptor binding site, and a T cell co-stimulatory molecule binding site, wherein two polypeptide chains have a structure represented by the formula: II₁-V_{L}-C_{L} II₂; and two polypeptide chains have a structure represented by the formula: V_{H1}-C_{H1}-V_{H2}-Fc; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; Fc is an immunoglobulin hinge region and C_{H2} and C_{H3} immunoglobulin heavy chain constant domains; II₁ and II₂ are each independently a heavy chain variable domain that specifically binds a T cell co-stimulatory molecule or are absent; wherein at least one of II₁ and II₂ is a heavy chain variable domain that specifically binds a T cell co-stimulatory molecule.

Some aspects described herein provide a binding protein comprising four polypeptide chains that form two tumor-associated antigen binding sites, a T cell receptor binding site, and a T cell co-stimulatory molecule binding site, wherein two polypeptide chains have a structure represented by the formula: V_{L}-C_{L} and one polypeptide chain has a structure represented by the formula: V_{H1}-C_{H1}-Fc; and one polypeptide chain has a structure represented by the formula: V_{H2}-V_{H3}-Fc; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH₁ heavy chain constant domain that specifically binds a tumor-associated antigen; V_{H2} is a heavy chain variable domain that specifically binds a T cell co-stimulatory molecule; V_{H3} is a heavy chain variable domain that specifically binds a T cell receptor binding site; Fc is an immunoglobulin hinge region and C_{H2} and C_{H3} immunoglobulin heavy chain constant domains.

Some aspects described herein provide a binding protein comprising three polypeptide chains that form two tumor-associated antigen binding sites, a T cell receptor binding site, and a T cell co-stimulatory molecule binding site, wherein the first polypeptide chain has a structure represented by the formula: V_{L}-C_{L}; and a second polypeptide chain has a structure represented by the formula: V_{H1}-C_{H1}-V_{H2}-Fc; and a third polypeptide chain has a structure represented by the formula: V_{H1}-V_{H3}-Fc; wherein: V_{L} is an immunoglobulin light chain variable domain that specifically binds a tumor-associated antigen; V_{H1} is an immunoglobulin heavy chain variable domain that specifically binds a tumor-associated antigen; C_{L} is an immunoglobulin light chain constant domain that specifically binds a tumor-associated antigen; C_{H1} is an immunoglobulin CH1 heavy chain constant domain that specifically binds a tumor-associated antigen; V_{H2} is a heavy chain variable domain that specifically binds a T cell receptor; V_{H3} is a heavy chain variable domain that specifically binds T cell co-stimulatory molecule; and Fc is C_{H2} and C_{H3} immunoglobulin heavy chain constant domains.

In some aspects, the heavy chain variable domain that specifically binds a T cell co-stimulatory molecule is an immunoglobulin heavy chain variable domain. In some aspects, the heavy chain variable domain that specifically binds a T cell co-stimulatory molecule is a single domain sequence. In particular aspects, the heavy chain variable domain that specifically binds a T cell co-stimulatory molecule is a nanobody. In particular aspects, the heavy chain variable domain that specifically binds a T cell co-stimulatory molecule is a camelid. In particular aspects, the heavy chain variable domain that specifically binds a T cell co-stimulatory molecule is a single-domain variable new antigen receptor.

In some aspects, the heavy chain variable domain that specifically binds a T cell receptor binding site is an immunoglobulin heavy chain variable domain. In some aspects, the heavy chain variable domain that specifically binds a T cell receptor binding site is a single domain sequence. In particular aspects, the heavy chain variable domain that specifically binds a T cell receptor binding site is a nanobody. In particular aspects, the heavy chain variable domain that specifically binds a T cell receptor binding site is a camelid. In particular aspects, the heavy chain variable domain that specifically binds a T cell receptor binding site is a single-domain variable new antigen receptor.

In some aspects, the Fc of the binding protein is from an IgG antibody for example IgG1, IgG2, IgG3, IgA1, and IgGA2.

In some aspects, the binding protein comprises a linker. The identity and sequence of amino acid residues in the linker may vary depending on the type of secondary structural element necessary to be achieved. For example, glycine, serine, and alanine are best for linkers having maximum flexibility. Some combination of glycine, proline, threonine, and serine are useful if a more rigid and extended linker is necessary. Any amino acid residue may be considered as a linker in combination with one or more other amino acid residues, which may be the same as or different as the first amino acid residue, to construct larger peptide linkers as necessary depending on the desired properties. In some aspects the binding protein comprises L₁, a linker positioned between C_{H1} and V_{H2} on the third polypeptide chain and L₂, a linker positioned between V_{H2} and the Fc on the third polypeptide chain, wherein L₁ and L₂ are each independently a linker or are absent. In some aspects, the binding protein comprises L₃, a linker positioned between C_{H1} and V_{H3} on the fourth polypeptide chain and L₄, a linker positioned between V_{H3} and Fc on the fourth polypeptide chain wherein L₃ and L₄ are each independently a linker or are absent. In some aspects, L₁, L₂, L₃, and L₄ are each independently a linker or are absent. In some aspects, the linker comprises the amino acid sequence TGGS (SEQ ID NO: 46). In some aspects, the linker comprises the amino acid sequence GGGGS (SEQ ID NO: 47). In some aspects, the linker comprises the amino acid sequence AAAYPYDVPDYGSGEGTSTGSGGSGGSGGA (SEQ ID NO: 48). In some aspects, the linker further comprises a hemagglutinin tag.

In some aspects, the binding protein comprises H₁, an immunoglobulin hinge region positioned between C_{H1} and V_{H2} on the third polypeptide chain and H₂, an immunoglobulin hinge region positioned between V_{H2} and the Fc on the third polypeptide chain, wherein H₁ and H₂ are each independently an immunoglobulin hinge region or are absent. In some aspects, the binding protein comprise H₃, an immunoglobulin hinge region positioned between C_{H1} and V_{H3} on the fourth polypeptide chain and H₄, an immunoglobulin hinge region positioned between V_{H3} and the Fc on the fourth polypeptide chain, wherein H₃ and H₄ are each independently an immunoglobulin hinge region or are absent. In some aspects, H₁, H₂, H₃, and H₄ are each independently an immunoglobulin hinge region or are absent.

In some aspects, the binding protein comprises a first and a second polypeptide chain having a structure represented by the formula:

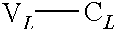

and a third polypeptide chain having a structure represented by the formula:

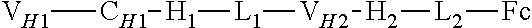

and a fourth polypeptide chain having a structure represented by the formula:

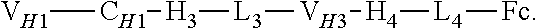

In other aspects, the binding protein comprises a first polypeptide chain having a structure represented by the formula:

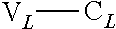

and a second polypeptide chain having a structure represented by the formula:

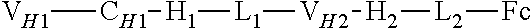

and a third polypeptide chain having a structure represented by the formula:

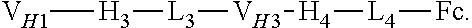

In other aspects, the binding protein comprises two polypeptide chains having a structure represented by the formula:

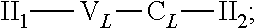

and two polypeptide chains having a structure represented by the formula:

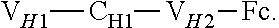

In other aspects, the binding protein comprises a first polypeptide chain having a structure represented by the formula:

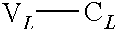

and a second polypeptide chain having a structure represented by the formula:

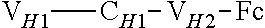

and a third polypeptide chain having a structure represented by the formula:

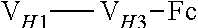

In some aspects, the binding protein comprises four polypeptide chains that form two tumor-associated antigen binding sites, a T cell receptor binding site, and a T cell co-stimulatory molecule binding site, wherein the polypeptide chains comprise
(a) the amino acid of sequences of SEQ ID NO: 1, SEQ ID NO: 2, and SEQ ID NO: 3;
(b) the amino acid of sequences of SEQ ID NO: 1, SEQ ID NO: 2, and SEQ ID NO: 4;
(c) the amino acid of sequences of SEQ ID NO: 1, SEQ ID NO: 9, and SEQ ID NO: 10;
(d) the amino acid of sequences of SEQ ID NO: 1, SEQ ID NO: 11, and SEQ ID NO: 12;
(e) the amino acid of sequences of SEQ ID NO: 1, SEQ ID NO: 13, and SEQ ID NO: 14;
(f) the amino acid of sequences of SEQ ID NO: 1 and SEQ ID NO: 19;
(g) the amino acid of sequences of SEQ ID NO: 1, SEQ ID NO: 22, and SEQ ID NO: 23;
(h) the amino acid of sequences of SEQ ID NO: 29, and SEQ ID NO: 30;
(i) the amino acid of sequences of SEQ ID NO: 31 and SEQ ID NO: 32;
(j) the amino acid of sequences of SEQ ID NO: 33 and SEQ ID NO: 34;
(k) the amino acid of sequences of SEQ ID NO: 35 and SEQ ID NO: 36; or
(l) the amino acid of sequences of SEQ ID NO: 37 and SEQ ID NO: 38.

In some aspects, the binding protein comprises three or four polypeptide chains that form two tumor-associated antigen binding sites, a T cell receptor binding site, and a T cell co-stimulatory molecule binding site, wherein the polypeptide chains comprise
(a) the amino acid of sequences of SEQ ID NO: 1, SEQ ID NO: 2, and SEQ ID NO: 3;
(b) the amino acid of sequences of SEQ ID NO: 1, SEQ ID NO: 2, and SEQ ID NO: 4;
(c) the amino acid of sequences of SEQ ID NO: 1, SEQ ID NO: 9, and SEQ ID NO: 10;
(d) the amino acid of sequences of SEQ ID NO: 1, SEQ ID NO: 11, and SEQ ID NO: 12;
(e) the amino acid of sequences of SEQ ID NO: 1, SEQ ID NO: 13, and SEQ ID NO: 14;
(f) the amino acid of sequences of SEQ ID NO: 1 and SEQ ID NO: 19;
(g) the amino acid of sequences of SEQ ID NO: 1, SEQ ID NO: 22, and SEQ ID NO: 23;
(h) the amino acid of sequences of SEQ ID NO: 29, and SEQ ID NO: 30;
(i) the amino acid of sequences of SEQ ID NO: 31 and SEQ ID NO: 32;
(j) the amino acid of sequences of SEQ ID NO: 33 and SEQ ID NO: 34;
(k) the amino acid of sequences of SEQ ID NO: 35 and SEQ ID NO: 36;
(l) the amino acid of sequences of SEQ ID NO: 37 and SEQ ID NO: 38;
(m) the amino acid of sequences of SEQ ID NO: 39 and SEQ ID NO: 40;
(n) the amino acid of sequences of SEQ ID NO: 41 and SEQ ID NO: 42; or
(o) the amino acid of sequences of SEQ ID NO: 45, and SEQ ID NO: 54.

In some aspects, the binding protein comprises three polypeptide chains that form two tumor-associated antigen binding sites, a T cell receptor binding site, and a T cell co-stimulatory molecule binding site, wherein the polypeptide chains comprise the amino acid of sequences of SEQ ID NO: 43, SEQ ID NO: 44, and SEQ ID NO: 45 or (q) the amino acid of sequences of SEQ ID NO: 1, SEQ ID NO: 11 and SEQ ID NO: 12. In one embodiment the binding protein comprises three polypeptide chains that form two tumor-associated antigen binding sites, a T cell receptor binding site, and a T cell co-stimulatory molecule binding site, comprises the amino acid of sequences of SEQ ID NO: 43, SEQ ID NO: 44, and SEQ ID NO: 45.

In some aspects provided herein is a binding protein comprising four polypeptide chains that form two tumor-associated antigen binding sites, a T cell receptor binding site, and a T cell co-stimulatory molecule binding site, wherein the polypeptide chains comprise amino acid sequences having at least 80%, or at least 90%, or at least 95%, or at least 99% sequence identity to any one of the amino acid sequences set forth in SEQ ID NOs: 1-56. In some aspects provided herein is a binding protein comprising four polypeptide chains that form two tumor-associated antigen binding sites, a T cell receptor binding site, and a T cell co-stimulatory molecule binding site, wherein the polypeptide chains comprise amino acid sequences having at least 80%, or at least 90%, or at least 95%, or at least 99% sequence identity to any one of the amino acid sequences set forth in SEQ ID NOs: 1-42. In some aspects provided herein is a binding protein comprising three polypeptide chains that form two tumor-associated antigen binding sites, a T cell receptor binding site, and a T cell co-stimulatory molecule binding site, wherein the polypeptide chains comprise amino acid sequences having at least 80%, or at least 90%, or at least 95%, or at least 99% sequence identity to any one of the amino acid sequences set forth in SEQ ID NOs: 1-56. In some aspects provided herein is a binding protein comprising three polypeptide chains that form two tumor-associated antigen binding sites, a T cell receptor binding site, and a T cell co-stimulatory molecule binding site, wherein the polypeptide chains comprise amino acid sequences having at least 80%, or at least 90%, or at least 95%, or at least 99% sequence identity to any one of the amino acid sequences set forth in SEQ ID NOs: 1-42.

In particular aspects provided herein are amino acid sequences with a conservative variant in which there are up to 10, up to 8, up to 5, and up to 3 amino acids substituted by amino acids having analogical or similar properties, compared to the amino acid sequence of the sequences disclosed herein.

The binding proteins of the disclosure may be prepared using domains or sequences obtained or derived from any human or non-human antibody, including, for example, human, murine, or humanized antibodies.

Some aspects of the present disclosure relate to an isolated nucleic acid sequence encoding a binding protein as described herein. The isolated nucleic acid sequence may be included in a vector.

In some aspects, the methods disclosed herein relate to treating a subject for cancer by administering an effective amount of a binding protein. The disclosure also provides a therapeutically effective amount of a binding protein for use in treating cancer in a subject. In one embodiment, there is provided a method for treating a subject for an inflammatory disease by administering an effective amount of a binding protein. In one embodiment, there is provided a binding protein or a pharmaceutical composition comprising the binding protein and a pharmaceutically acceptable carrier, for use as a medicament. In another embodiment, there is provided a binding protein or a pharmaceutical composition comprising the binding protein and a pharmaceutically acceptable carrier, for use in the treatment of cancer. In another embodiment, there is provided a binding protein or a pharmaceutical composition comprising the binding protein and a pharmaceutically acceptable carrier, for use in the treatment of an inflammatory disease. In another embodiment, there is provided the use of a binding protein or a pharmaceutical composition comprising the binding protein and a pharmaceutically acceptable carrier, in the manufacture of a medicament for use in the treatment of an inflammatory disease. In a further embodiment, there is provided the use of a binding protein or a pharmaceutical composition comprising the binding protein and a pharmaceutically acceptable carrier, in the manufacture of a medicament for use in the treatment of cancer.

In some aspects, the cancer includes B-cell malignancies, including chronic lymphocytic leukemia, diffuse large B-cell lymphoma, follicular lymphoma, and mantle cell lymphoma.

In some aspects, the methods disclosed herein relate to treating a subject for an inflammatory disease where B cells are involved by administering an effective amount of a binding protein. The disclosure also provides a therapeutically effective amount of a binding protein for use in treating an inflammatory disease where B cells are involved in a subject. In the method of treatment of the present disclosure, the binding proteins can preferentially activate a subset of T cells in the subject. The subset of T cells can be CD8+ T cells. The CD8+ T cells can be preferentially activated as compared to CD4 T cells. The preferential activation of CD8+ T cells reduces engagement with tumor promoting T cells and CD4+ T cells which produce the majority of cytokines that cause release syndrome (CRS). Additionally, the preferential engagement of the CD8+ T cells induce pyroptosis.

The activation of T cells through methods of the present disclosure can be determined by measuring the percentage of surface interleukin-2 receptor alpha chain-positive (CD25+) T cells. The percentage of surface CD25+ T cells that are CD8 T cells can be higher than the percentage of surface CD25+ T cells that are CD4 T cells. In particular aspects, activation of T cells can be determined by the percentage of CD69+/CD25+ T cells. In particular aspects, activation of T cells can be determined by measuring levels of cytokines released by activated T cells.

The method of treatment of the present disclosure can result in reduced engagement of regulatory T cells (Tregs), increased cytolytic activity, and/or reduced incidence of cytokine release syndrome (CRS) relative to that resulting from bispecific T-cell engager (BiTEs) previously known in the art. The disclosure also provides a therapeutically effective amount of a binding protein for use in reducing engagement of regulatory T cells (Tregs), increasing cytolytic activity, and/or reducing incidence of cytokine release syndrome (CRS) relative to that resulting from bispecific T-cell engager (BiTEs) previously known in the art.

In view of the present disclosure, the methods and compositions described herein can be configured by the person of ordinary skill in the art to meet the desired need.

In some aspects, the inflammatory disease or inflammatory disease where B cells are involved is selected from rheumatoid arthritis, multiple sclerosis, inflammatory bowel disease, psoriasis, pemphigus, lupus profundus, sceleroderma, discoid lupus erythematosus, systemic lupus erythematosus, Sjögren's syndrome, atopic dermatitis and allergic contact dermatitis.

### EXAMPLES

The Examples that follow are illustrative of specific aspects of the disclosure, and various uses thereof. They are set forth for explanatory purposes only and should not be construed as limiting the scope of the disclosure in any way.

### Example 1: Binding Protein Structure and Cytolytic and T-Cell Activity

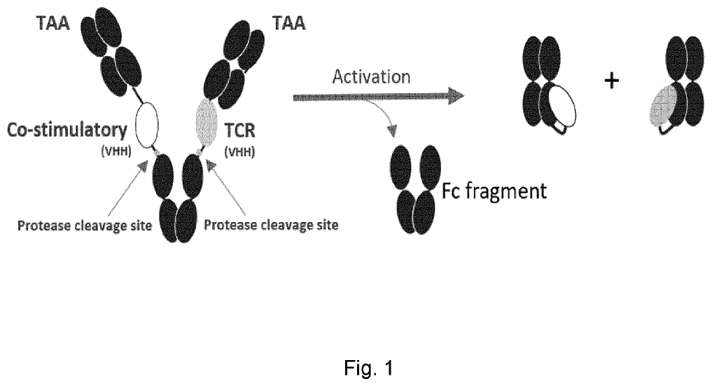

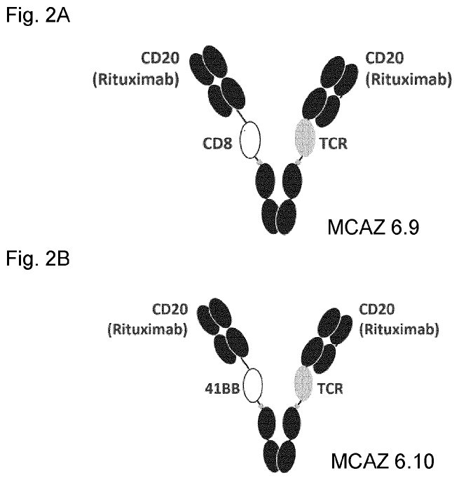

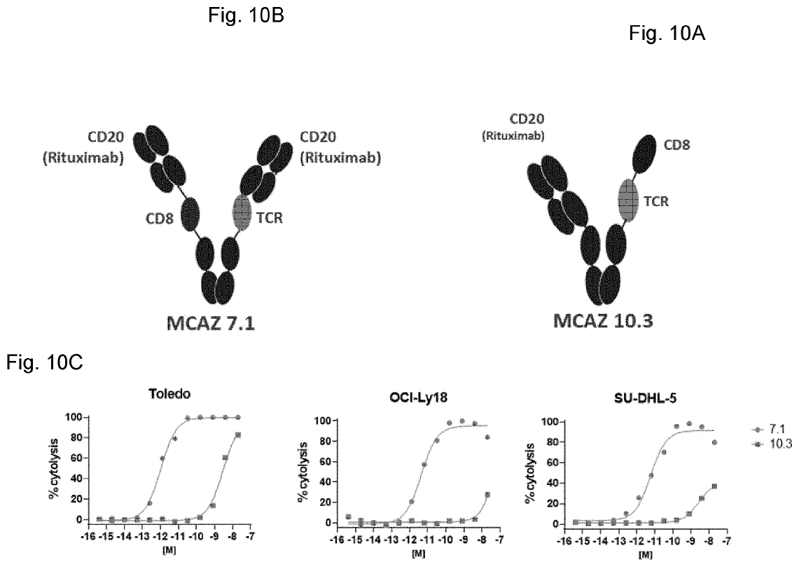

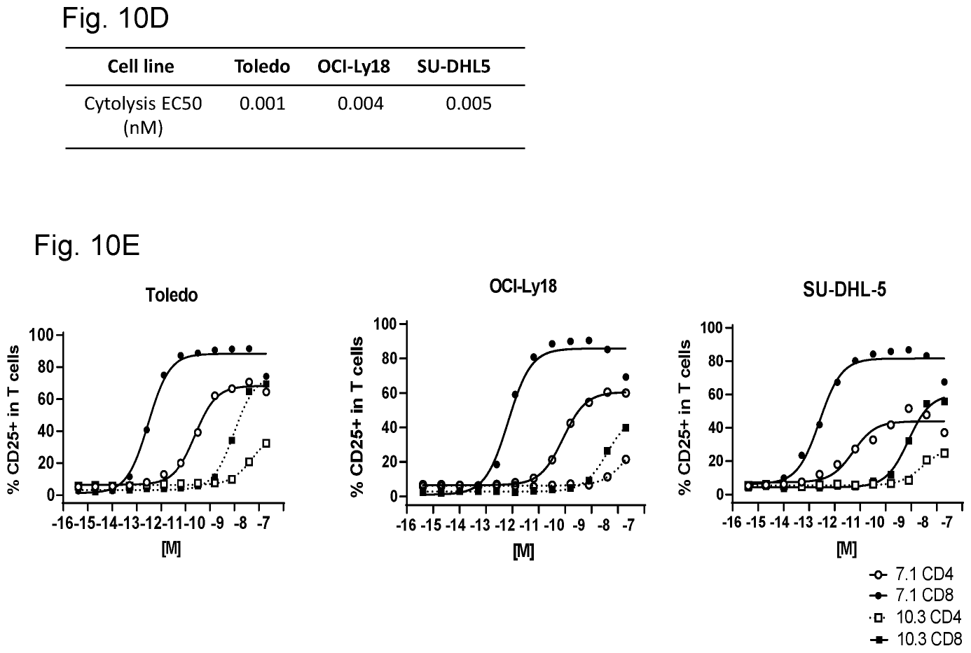

A novel class of binding proteins engineered to improve safety and increase efficacy is described herein. A multitude of binding protein formats were tested to determine the placement of the TCR binding domain, the placement of the T-cell co-stimulatory domain, the linker size and cleavability and the Fc portion to be used. FIG. 1 shows a representative structure for these binding proteins. As shown in FIGS. 2A and 2B, binding proteins MCAZ 6.9 and MCAZ 6.10 respectively were generated to include two CD20 binding sites, a CD8 co-stimulatory binding arm (FIG. 2A) and CD137 binding arm (FIG. 2B), a TCR binding domain and two protease cleavage sites. Similarly, MCAZ 7.1 was generated to include two CD20 binding sites, a CD8 co-stimulatory binding arm, and a TCR binding domain, but did not include the two protease cleavage sites (FIG. 10A).

MCAZ 6.9 and MCAZ 6.10 binding proteins were assessed for in vitro cytolysis on CD20+ B cell lines Daudi, Ramos and Raji. B cells and purified pan T cells were incubated with the B cell lines at an E:T ratio of 5:1 for 4 days and % cytolysis was measured by flow cytometry. Cytolysis for MCAZ 7.1 was assessed on CD20+ B cell lines Toledo, Oci-LY18, and SU-DHL5 (respectively expressing 12420, 20244, and 27152 CD20 antigen per cell). B cell lines were CTV stained and incubated PBMCs at an E:T ratio of 5:1 for 4 days. % cytolysis was measured by flow cytometry.

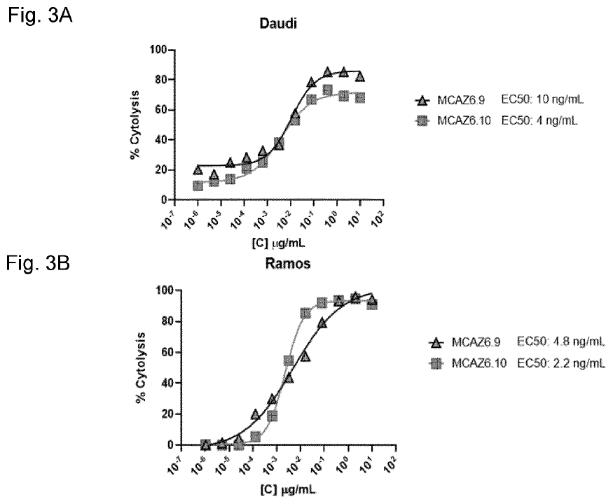

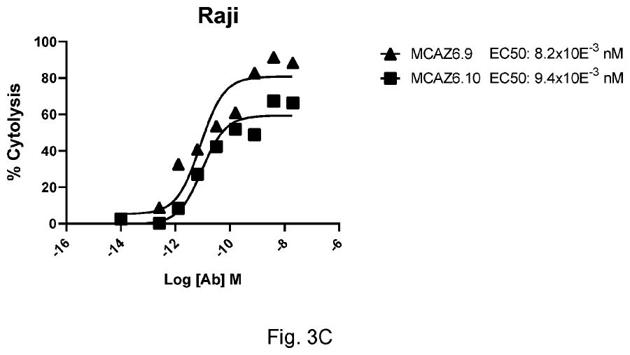

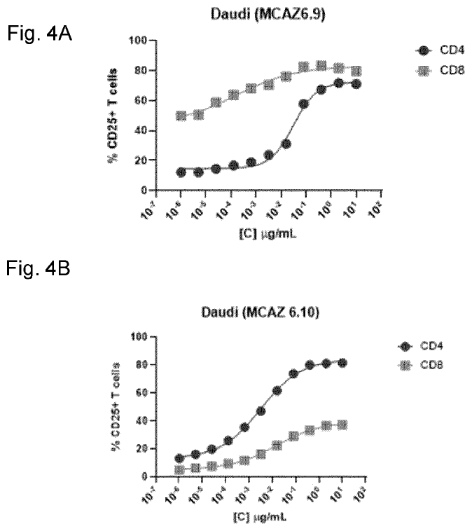

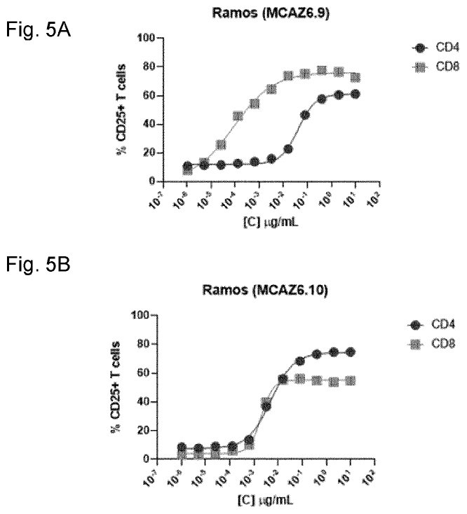

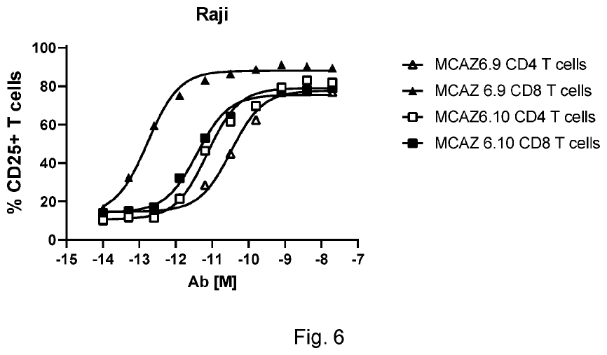

MCAZ 6.9, MCAZ 6.10, and MCAZ 7.1 binding proteins induced a strong in vitro cytolytic activity (FIGS. 3A-3C, FIGS. 10C & 10D). Cytolysis was associated with potent T cell activation that was biased to CD8 T cells for MCAZ 6.9 and MCAZ 7.1 (FIGS. 4A, 5A, 6 and FIG. 10E) and had CD4 biased profile or equivalent CD4/CD8 profiles for MCAZ 6.10 (FIGS. 4B, 5B, and 6).

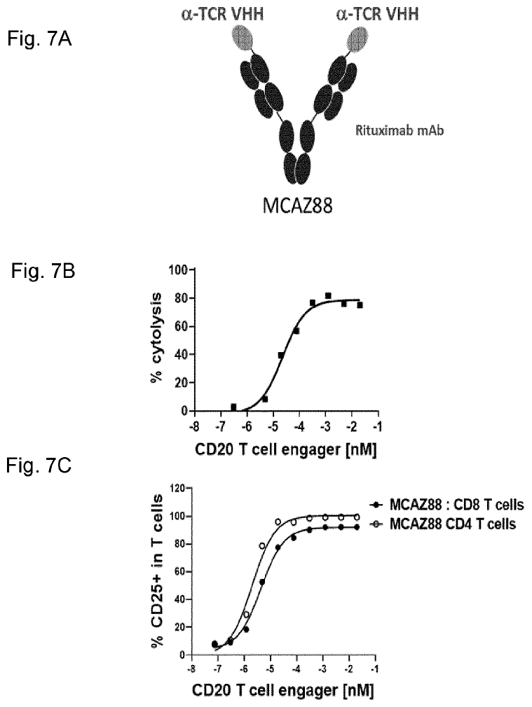

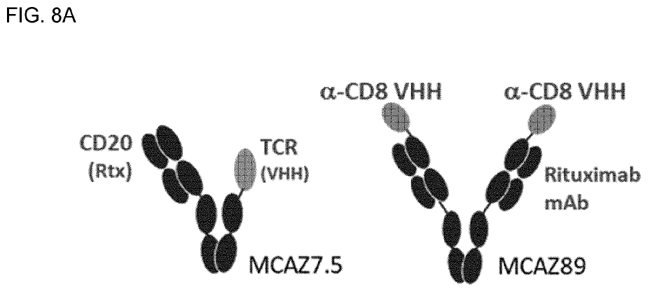

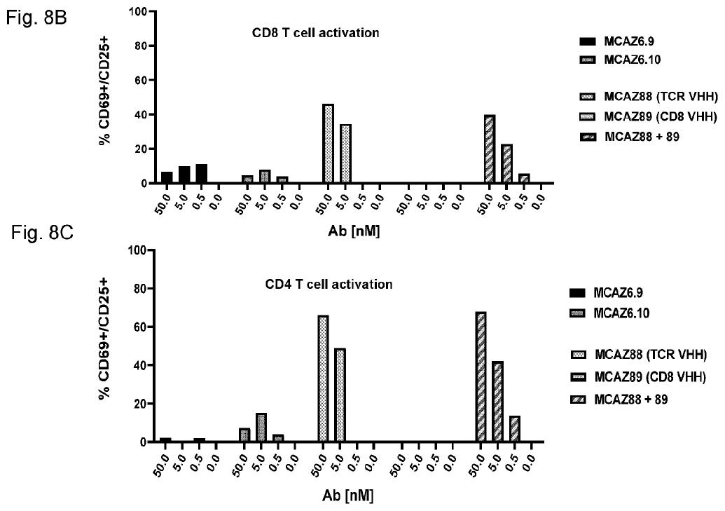

To determine the optimal placement of the TCR binding domain, MCAZ 88, MCAZ 7.5, MCAZ 89 were each generated with a unique placement of the TCR binding domain or absence of the TCR binding domain. MCAZ 88 included two TCR binding domains on the end of each of the variable heavy domains (FIG. 7A), MCAZ 7.5 had the TCR binding domain placed in the hinge portion of the binding protein (FIG. 8A), while the MCAZ 89 binding protein did not include any TCR binding domains (FIG. 8A).

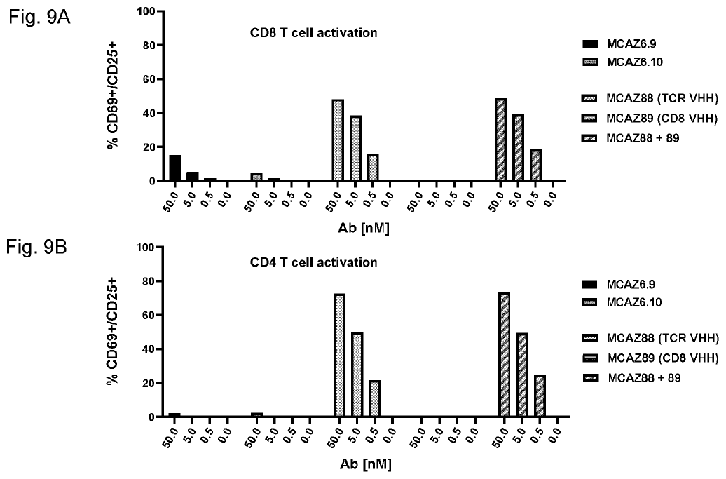

To test cytolysis for MCAZ 88, induced cytolysis was assessed on Raji cell line (CD20+, 85371 antigen per cell). B cells and PBMCs were incubated with the B cell line at an E:T ratio of 5:1 for 4 days and % cytolysis was measured by flow cytometry. CD4 and CD8 T cell activation profiles were also assessed for MCAZ88, MCAZ7.5, and MCAZ89 binding proteins by measuring the % of surface CD25+ cells and % CD69+/CD25+ surface expression. The results demonstrated that the presence of the two TCR-binding domains in the absence of the T-cell co-stimulatory domain were sufficient to induce cytolysis (FIG. 7B). However, both the absence of a TCR binding domain and the placement of the TCR binding domain at the top of the binding protein resulted in non-specific T cell activation (FIG. 7C, FIG. 8B, FIG. 8C, FIG. 9). Non-specific T-cell activation was strongly decreased only when the TCR binding domain was in the hinge region of the binding protein (FIG. 8B, FIG. 8C).

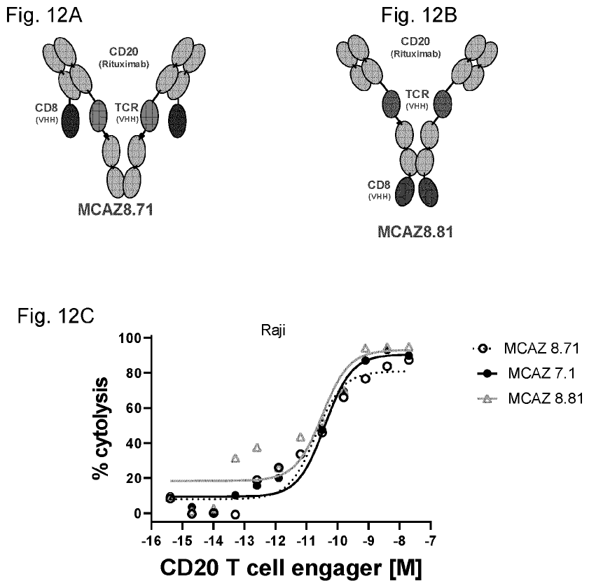

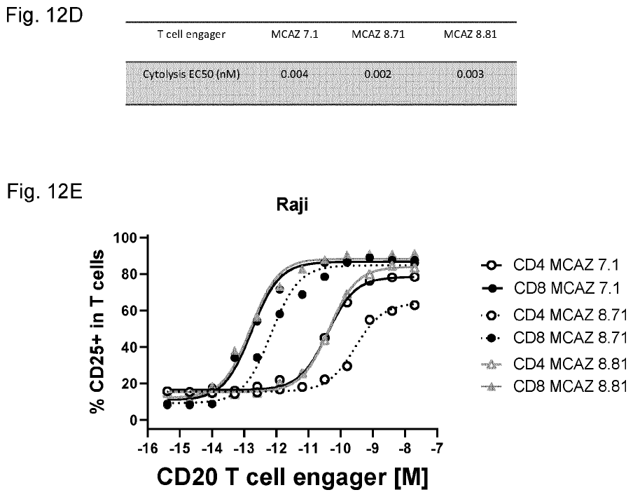

The binding proteins MCAZ 8.71 and MCAZ 8.81 were each generated with two T cell co-stimulatory molecule in a unique placement on the binding protein to evaluate the impact of CD8 positioning on the binding protein function (FIGS. 12A-12B). Cytolysis was assessed on CD20+ B Raji cell line (expressing 85000 CD20 antigen per cell). B cell lines were CTV stained and incubated with PBMCs at an E:T ratio of 5:1 for 4 days. % cytolysis was measured by flow cytometry. CD4 and CD8 T cell activation profile were assessed as % CD25 expressing cells. Despite the different positions of the T cell co-stimulatory molecule or the presence of an extra T-cell co-stimulatory molecule in the binding protein, both MCAZ 8.71 and MCAZ 8.81 showed similar cytolysis (FIGS. 12C-12D). Cytolysis was associated with potent T-cell activation that was biased to CD8 T cells for MCAZ 8.71 and MCAZ 8.81 (FIG. 12E). These results demonstrate that the position of the co-stimulatory molecule was flexible within the binding protein, however extra molecules did not increase potency of the binding protein.

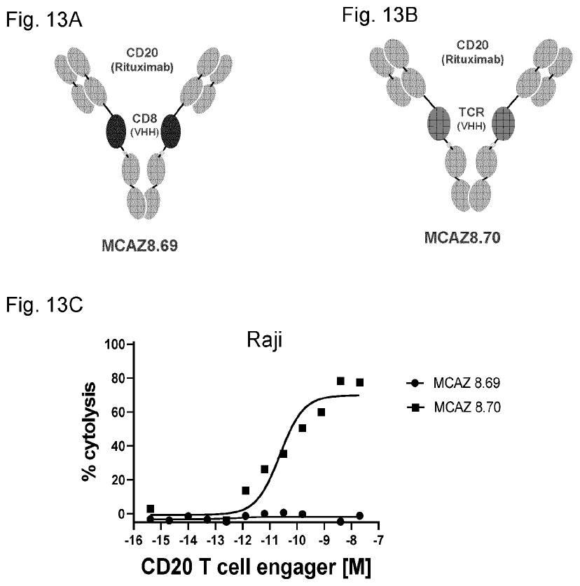

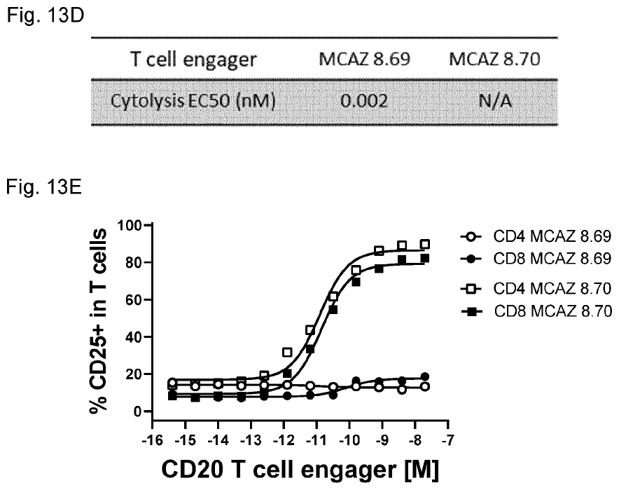

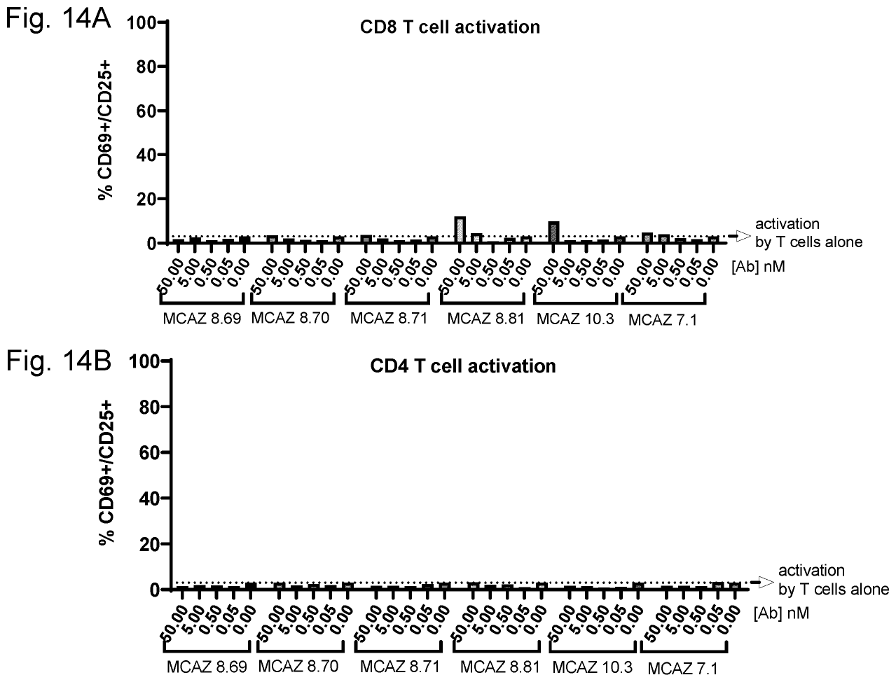

To test the placement of the T-cell co-stimulatory domain and the T-cell binding domain in relation to each other, binding proteins of different orientations were tested. MCAZ 10.3 was generated to include both the CD8 domain and the TCR binding domain on the same polypeptide (FIG. 10C). The T-cell co-stimulatory domain and the T-cell binding domain were also tested on separate binding proteins. Specifically, MCAZ 8.69 was generated as a binding protein that only included the T-cell co-stimulatory molecule and MCAZ 8.70 was generated as a binding protein that only included the TCR binding domain (FIG. 13A). Cytolysis was assessed on CD20+ B Raji cell line, Toledo, Oci-LY18, and SU-DHL5 cell line. B cell lines were CTV stained and incubated with PBMCs at an E:T ratio of 5:1 for 4 days. % cytolysis was measured by flow cytometry. CD4 and CD8 T cell activation profiles were assessed as % CD25 expressing cells. The results demonstrated that the ideal position of the CD8 domain and the TCR binding domain was on separate arms, but present in the same binding protein for cytolysis and T cell activation (FIGS. 10C-10E and FIG. 13).

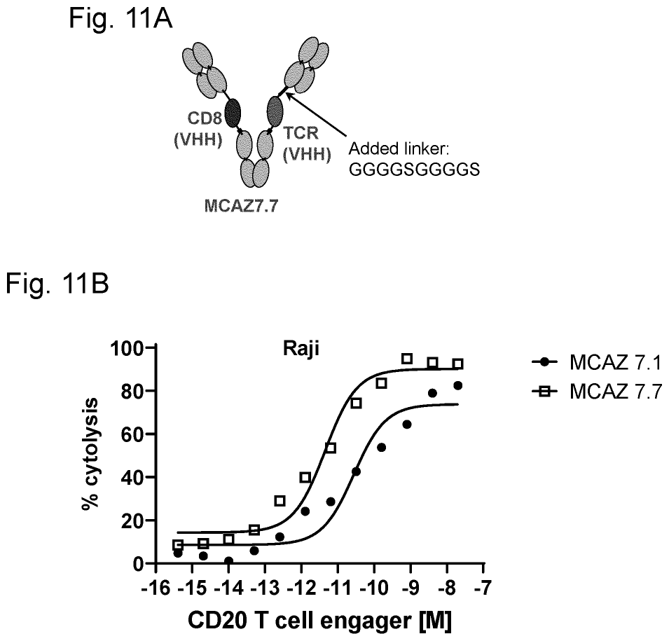

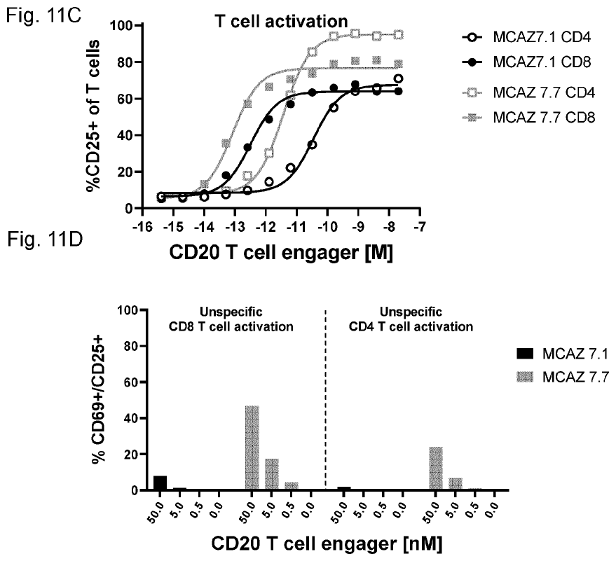

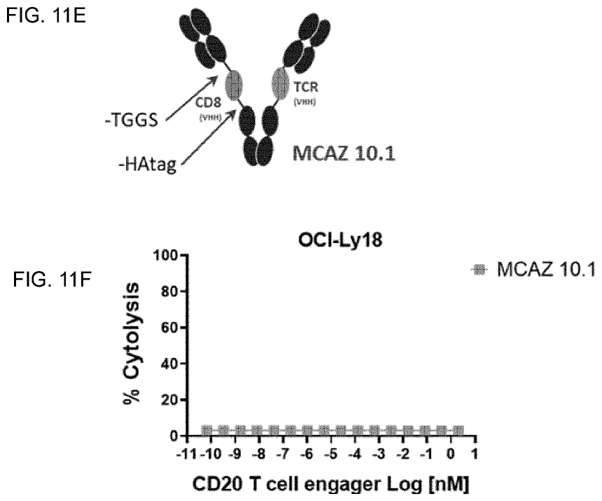

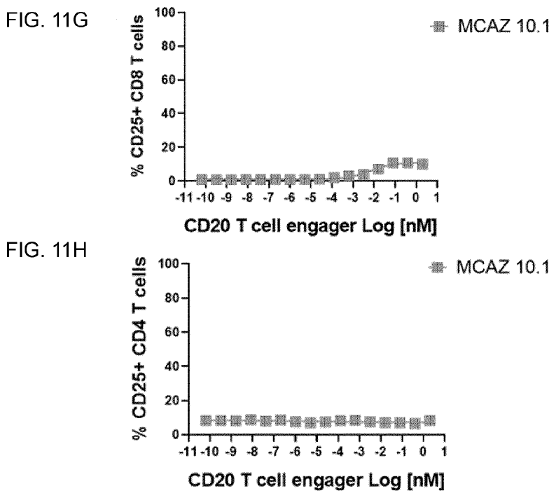

Modified linker lengths were tested to determine the optimal linker for stability and function of the binding protein. MCAZ 7.7 was generated with a modified longer linker (GGGGSGGGGS) positioned between the CD20 binding domain and the TCR-binding domain (FIG. 11A) as compared to MCAZ 7.1 that had a linker (T) positioned between the CD20 binding domain and the TCR-binding domain. MCAZ 10.1 was generated with a more rigid CD8 arm as compared to MCAZ 7.1. Specifically, the (TGGS (SEQ ID NO: 46)) linker positioned between the CD20 binding domain and the CD8 domain was removed and the linker positioned between the CD8 domain and the Fc domain was switched from (AAAYPYDVPDYGSGEGTSTGSGGSGGSGGA (SEQ ID NO: 48)) in MCAZ 7.1 to (G) in MCAZ 10.1 (FIG. 11E). Additionally, MCAZ 10.1 was generated with the linker positioned between the CD20 binding domain and the TCR-binding domain deleted as compared to MCAZ 7.1 that had a linker (T) and a shortened linker (G) positioned between the TCR-binding domain and the Fc as compared to MCAZ 7.1 that had a linker (GEGTSTGSGGSGGSGGA (SEQ ID NO: 49)). For MCAZ 7.7, cytolysis was assessed on CD20+ Raji B cell line (expressing 85000 CD20 antigen per cell). B cell lines were CTV stained and incubated PBMCs at an E:T ratio of 5:1 for 4 days. % cytolysis was measured by flow cytometry. For MCAZ 10.1, cytolysis was assessed on OCI-Ly18 B cell line. B cell lines were CTV stained and incubated PBMCs at an E:T ratio of 5:1 for 3 days. % cytolysis was measured by flow cytometry. T cell activation profile were assessed as % CD25 expressing cells. For MCAZ 7.7, unspecific and specific T cell activation was assessed by having binding proteins plate-bound and 1.5e5 purified T cells were added, in the absence of tumor cells and associated proteases. After 48 h incubation, non-specific CD8 and CD4 T cell activation levels were assessed by flow cytometry as % CD69+/CD25+ surface expression. The modified longer linker in MCZA 7.7 increased levels of cytolysis as compared to MCZA 7.1, however the longer linker in MCZA 7.7 resulted in non-specific T cell activation. The removal of the linker in MCAZ 10.1 inhibited the function of the binding protein and resulted in minimal cytolysis and T cell activation.

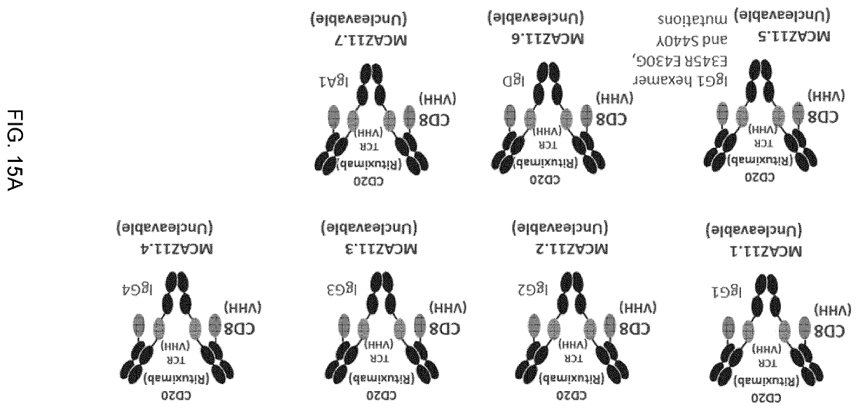

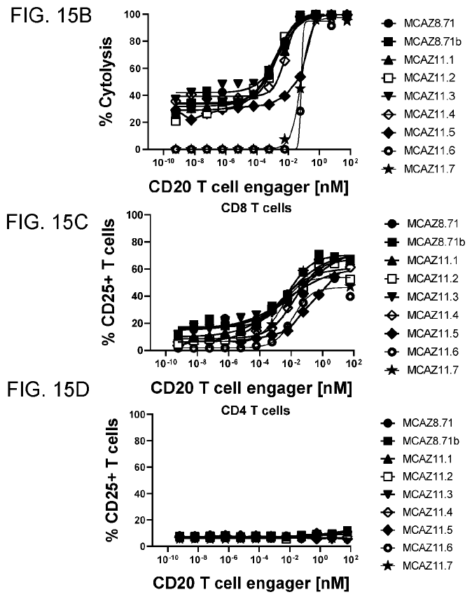

Various binding proteins as illustrated in FIG. 15A were generated to test different uncleavable Fc Regions including IgG1 (MCAZ 11.1), IgG2 (MCAZ 11.2), IgG3 (MCAZ 11.3), IgG4 (MCAZ 11.5), mutated IgG1 (MCAZ 11.5), IgD (MCAZ 11.6) and IgA1 (MCAZ 11.7). The cytolytic activity of the binding proteins was tested at 72 h on OCI-Ly18 B cell line, with PBMCs at E:T ratio of 5:1. CD8 and CD4 T cell activation profiles were assessed by measuring the % of surface CD25+ T cells. The binding proteins with the IgG Fc regions were most effective at cytolysis and inducing T cell activation (FIGS. 15B-15D).

Based on the above experiments, the format illustrated in for example MCAZ 7.1 was found to provide optimal cytolysis and inducing T cell activation.

### Example 2: In Vitro Testing of Binding Proteins as Compared to a Broad CD3×CD20 Bivalent Engager

The activity of the novel binding proteins disclosed herein were compared to a broad CD20 bivalent engager established as effective in clinical treatment.

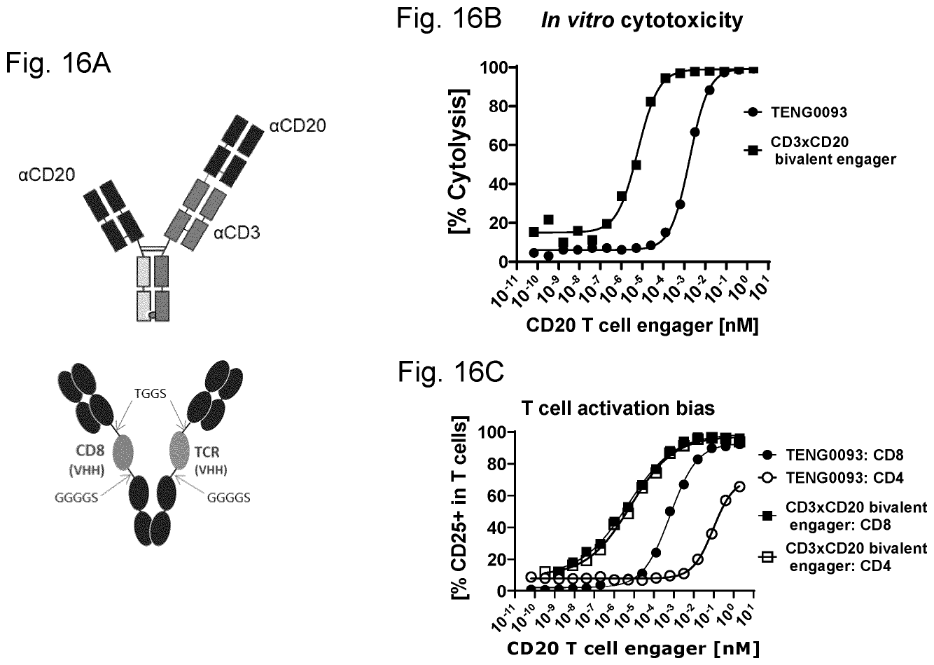

The activity of a variant of MCAZ 7.1 and a broad CD3×CD20 bivalent engager was compared (FIG. 16A). Specifically, the variant included a linker between the CD20 binding domain and the CD8 domain and a linker between the CD20 binding domain and the TCR domain (TGGS (SEQ ID NO: 46)), as well as a linker between the CD8 domain and the Fc region and the TCR domain and the Fc domain (GGGGS (SEQ ID NO: 47)). The binding protein and bivalent engager were incubated with target CD20+ cells for 72 h at an E:T ratio of 5:1 with PBMCs from a healthy donor. The MCAZ7.1 variant induced similar E max cytolysis (˜pM EC50) but with a distinct significant CD8-biased activation profile (FIG. 16B).

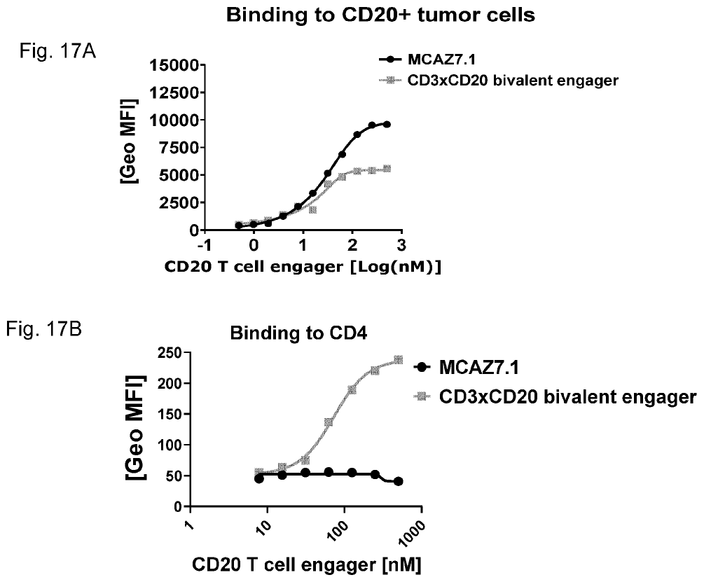

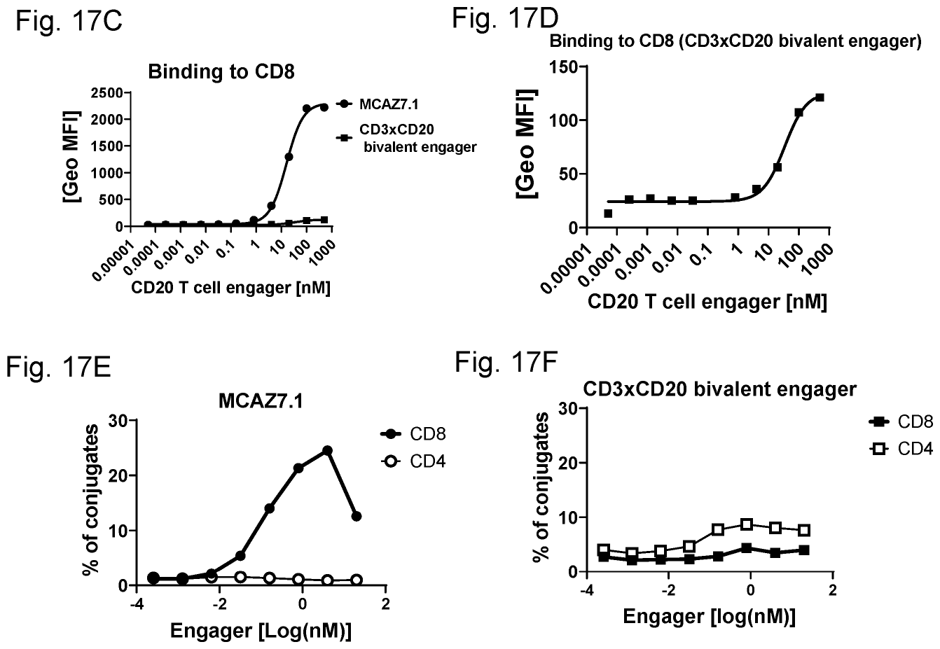

The binding to CD8 T cells of the variant of MCAZ 7.1 and the CD3×CD20 bivalent engager was compared. The MCAZ7.1 variant binding profile was assessed on CD20+ tumor B cell line (OCI-Ly-18), on purified CD4 and CD8 T cells from PBMCs from healthy donors. To assess CD3×CD20 bivalent engager induced B cell:T cell association, OCI-Ly18 tumor cells were CTV stained and mixed at a 1:1 ratio with pan-T cells from a healthy donor and incubated 1 h at room temperature. The % of CD8-B cell conjugates and CD4-T cell conjugate was then assessed by flow cytometry after staining of CD4 and CD8 T cells after incubation with the MCAZ7.1 variant and the CD3×CD20 bivalent engager. The results demonstrated that compared to a therapeutically effective broad CD3×CD20 bivalent engager, the MCAZ7.1 variant exhibited strong binding to CD8 T cells and induced preferential association of CD20+ tumor cells with CD8 T cells (FIGS. 17A-17F).

A 3D-spheroid model was used to determine cytolytic activity of the MCAZ 7.1 variant as compared to the CD3×CD20 bivalent engager. GFP-expressing CD20+ B cell line (TMD8, 100 000 CD20/cell) were plated in low adherent plate to form 3D-spheroids for 72 h. Then, purified panT cells were added at a 15:1 E:T ratio and co-incubated with no engager, TENG0093, or CD3×CD20 bivalent engager for 96 h. GFP+ TMD8 cells were quantified using a Cellinsight CX7 HCS Platform imager. The variant of MCAZ 7.1 provided potent CD20+ cell killing comparable to the CD3×CD20 bivalent molecule (FIG. 20B).

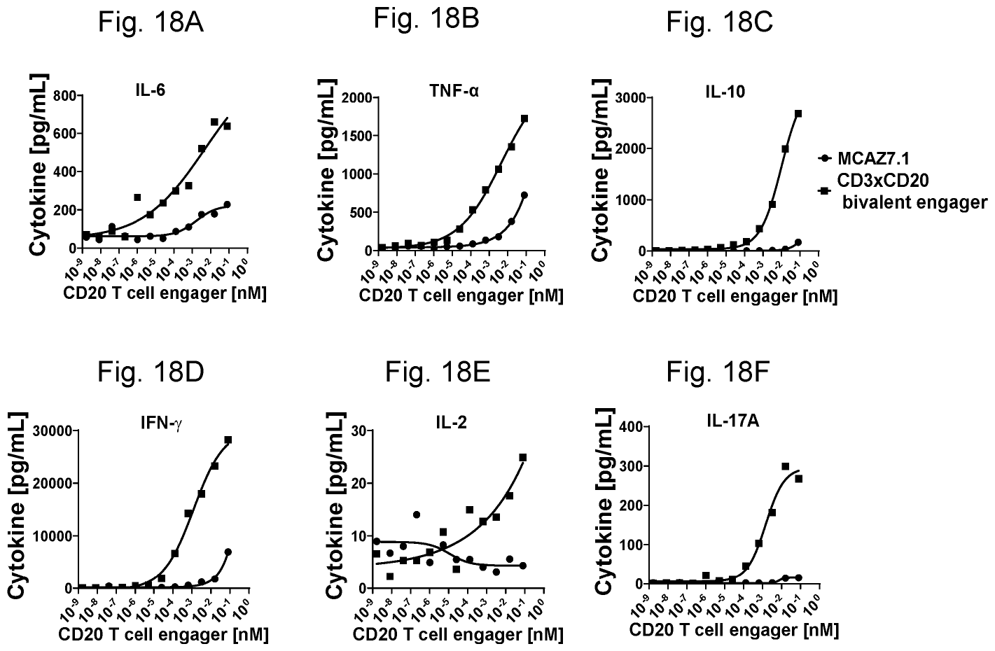

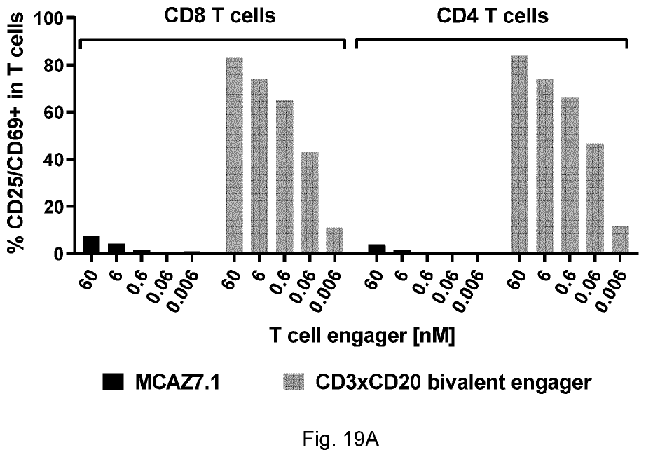

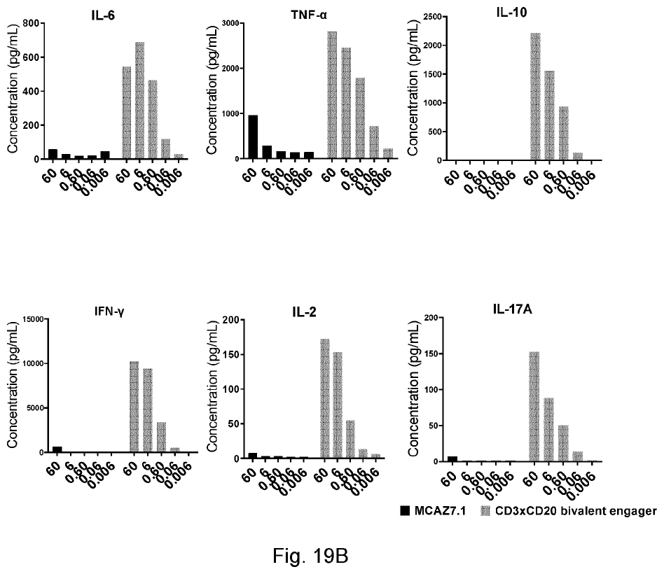

To assess the inflammatory cytokine release of the MCAZ7.1 binding protein compared to the CD3×CD20 bivalent engager, MCAZ7.1 and the CD3×CD20 bivalent engager were incubated with OCI-Ly18 B cell line and PBMCs at an E:T ratio of 5:1 for 72 h. Supernatants were harvested and analyzed by multiplex assay to measure the concentrations of released pro-inflammatory cytokines: (FIG. 18A) IL-6, (FIG. 18B) TNF-α, (FIG. 18C) IL-10, (FIG. 18D) IFN-g, (FIG. 18E) IL-2, and (FIG. 18F) IL-17A. CD8-specific engagement of MCAZ7.1 provided similar Emax killing of CD20+ tumor cells as the CD3×CD20 bivalent engager, but cytolysis was associated with significantly lower pro-inflammatory cytokine release than the CD3+ T cell engagement by the CD3×CD20 bivalent engager.

### Example 3: In Vivo Efficacy in B Cell Lymphoma in a Humanized NSG Mouse Model

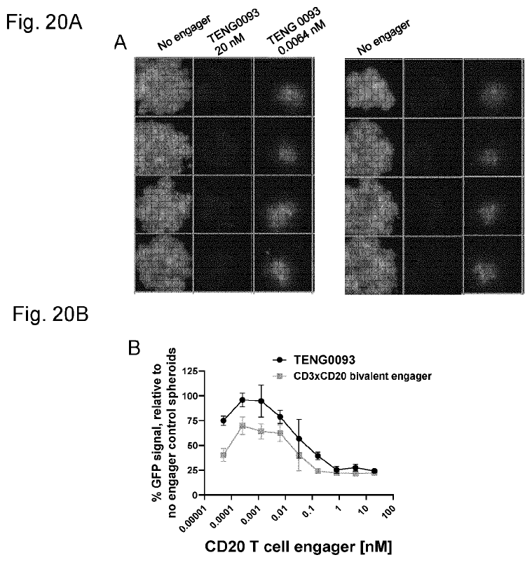

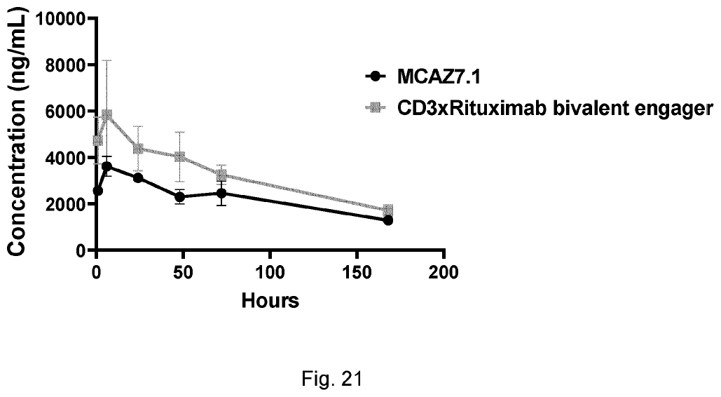

The pharmacokinetics of MCAZ 7.1 was assessed after a single dose. Specifically, NSG mice humanized with panT cells were injected with 0.5 mg/kg of T cell engager and systemic concentrations of T cell engagers were measured after 1 h, 6 h, 24 h, 48 h, 72 h, and 168 h. The results demonstrate that the MCAZ 7.1 binding protein was stable in vivo (FIG. 21).

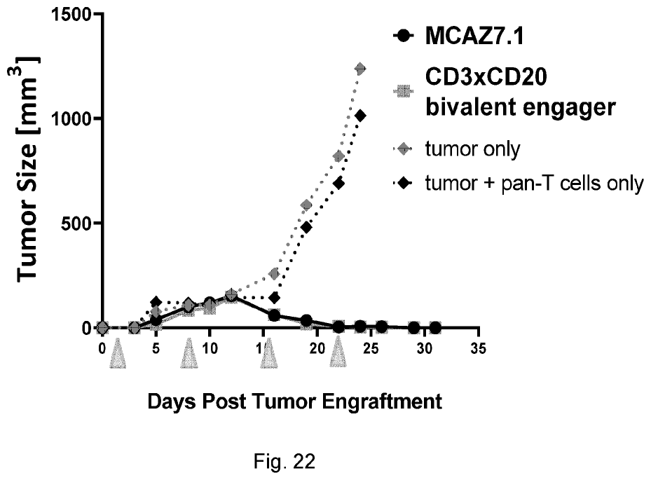

To test efficacy of tumor growth inhibition of the MCAZ 7.1 binding protein, NSG mice were engrafted with panT cells two days prior to the study start. 5e6 OCI-Ly18 CD20+ tumor cell lines were implanted s.c. at day 0 and animals were dosed i.p. with 1 mg/kg of MCAZ7.1 or the CD3×CD20 bivalent engager at day 2, followed by weekly dosing at the same concentration as indicated by the grey arrows under the X axis. MCAZ 7.1 and the CD3×CD20 bivalent engager showed similar efficacy and complete tumor growth inhibition (FIG. 22).

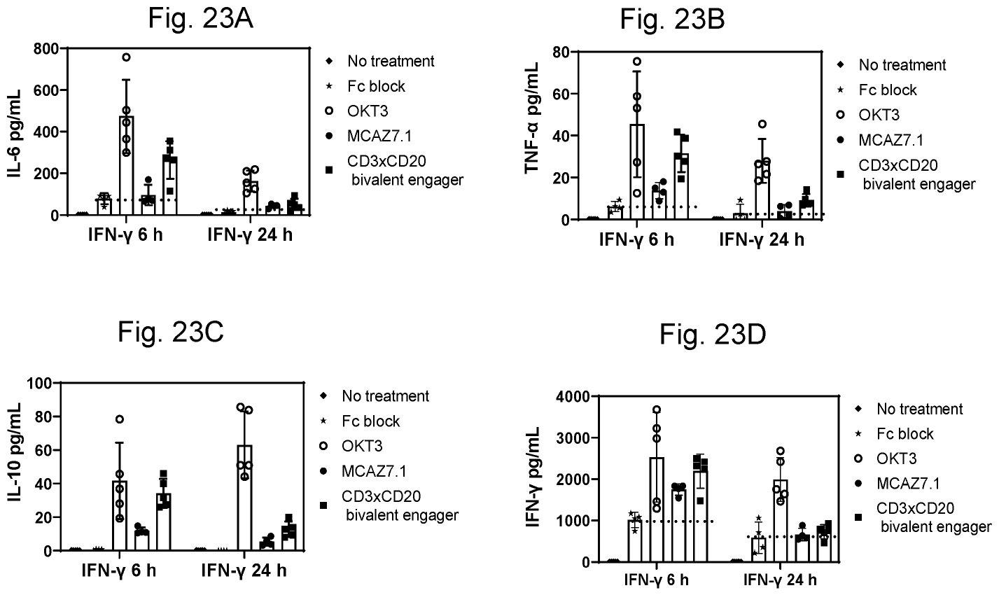

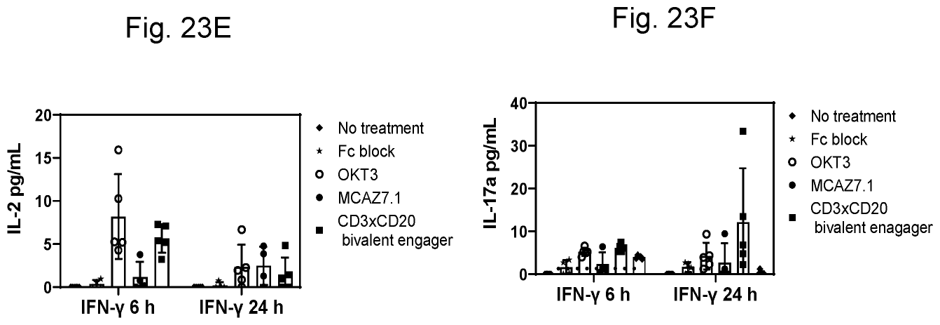

To assess in vivo cytokine release of MCAZ 7.1 compared to the CD3×CD20 bivalent engager, NSG mice were irradiated (2.3 Gy) before engraftment with 10e6 PBMCs (i.p.) after 48 h, treated with Fc block (400 mg/mouse i.p.), and 24 h later treated with 2 mk/kg for OKT3 antibody (i.p.) or the indicated binding protein at 1 mg/kg (i.p.). Blood was harvested at 6 h and 24 h post injection for assessment of cytokine concentration by multiplex assay. In vivo cytokine release assessment in a CRS (cytokine release syndrome) model confirmed MCAZ 7.1 induced lower cytokine release compared to broad CD3+ T cell engagement induced by the CD3×CD20 bivalent molecule (FIGS. 23A-23F).

### Example 4: Binding Proteins In Vitro Activity on PBMCs from Non-Hodgkin Lymphoma Donors

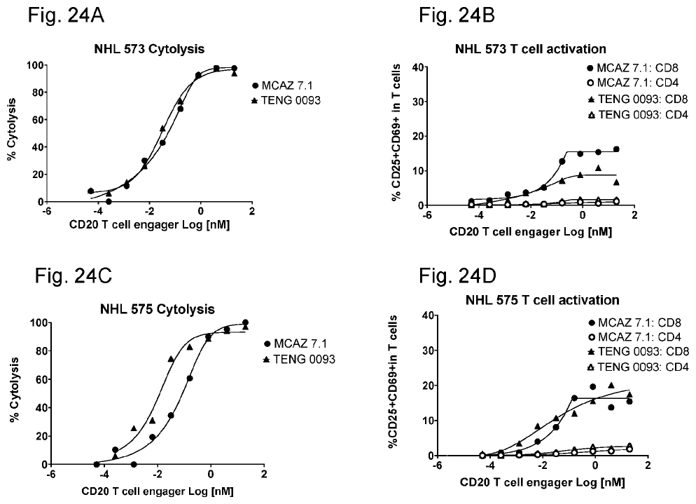

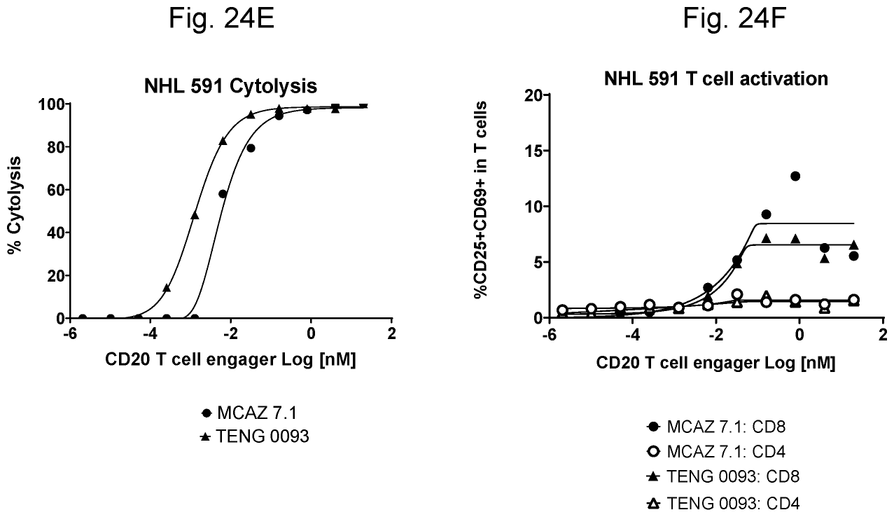

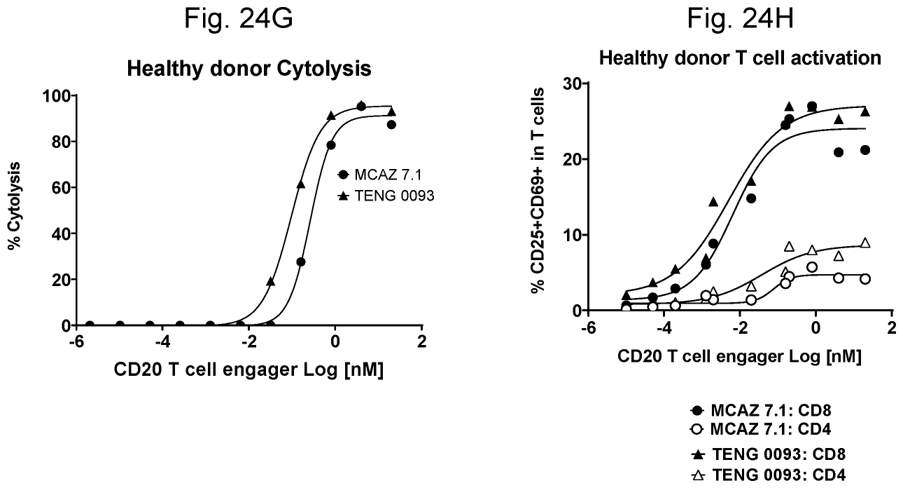

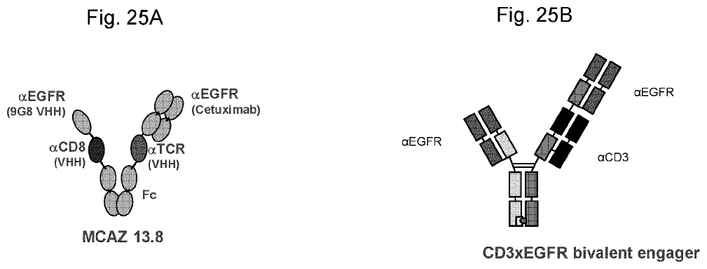

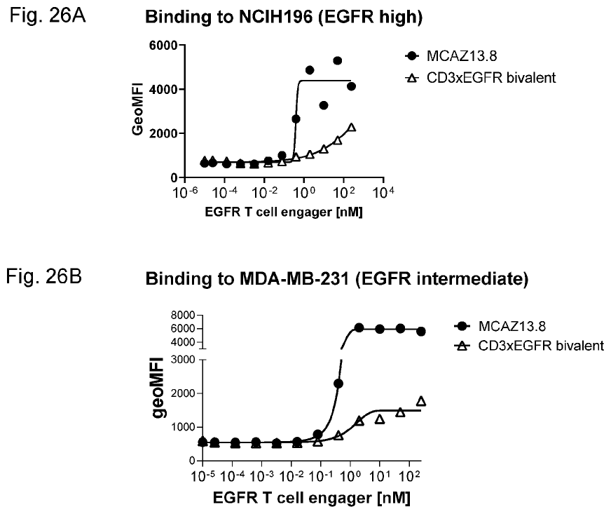

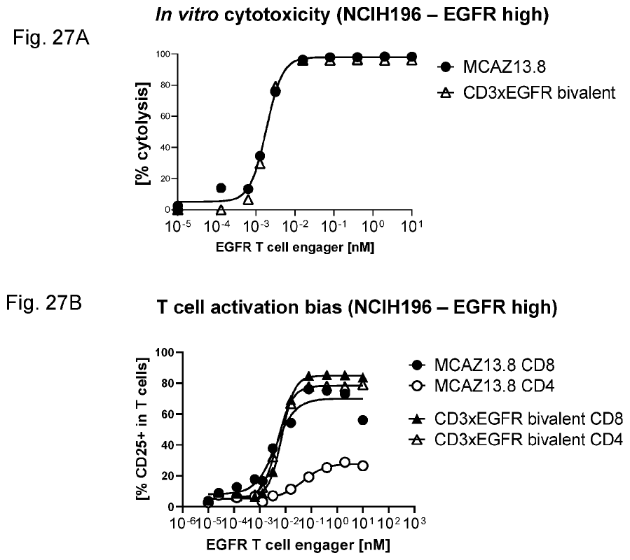

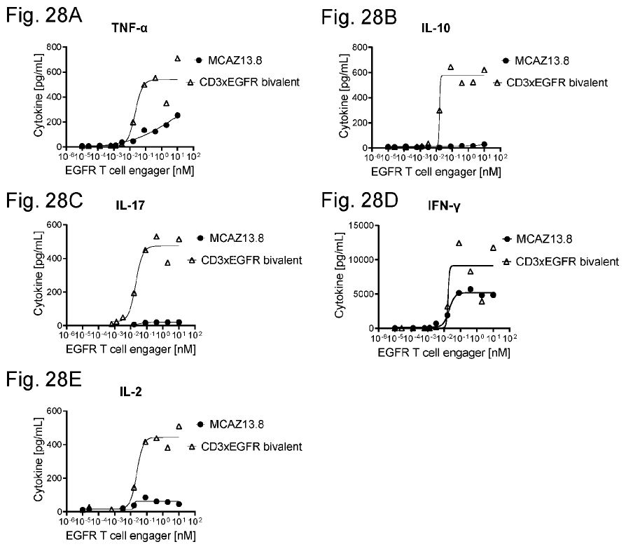

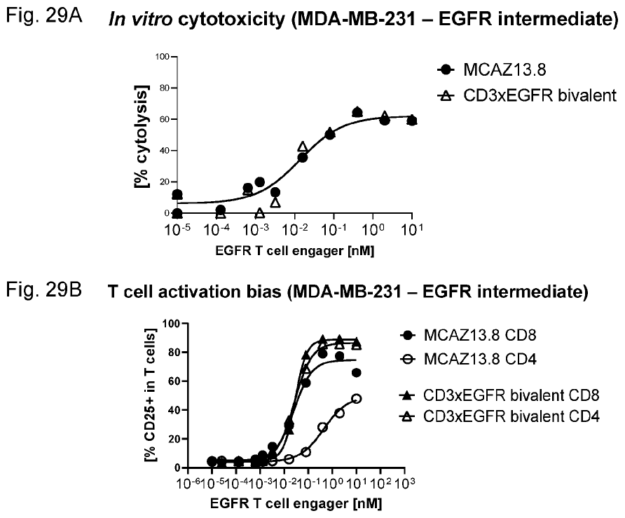

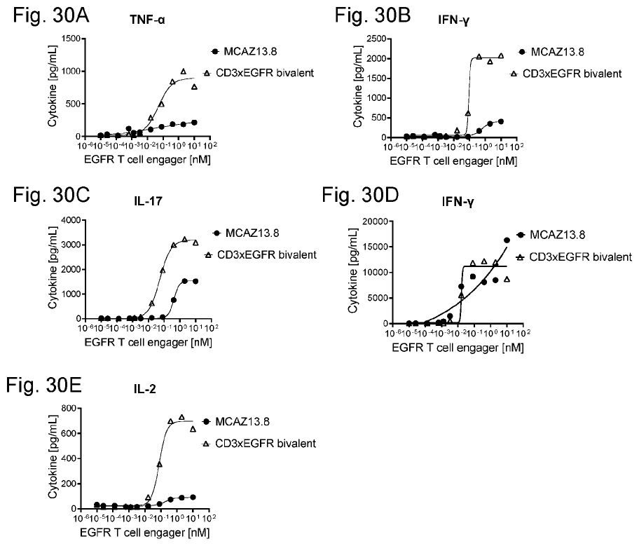

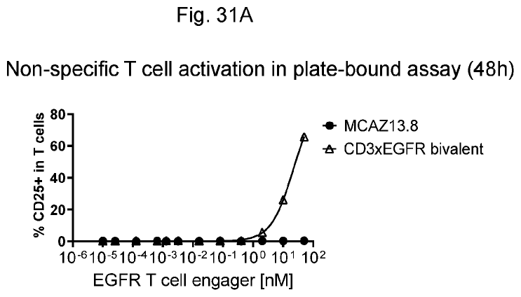

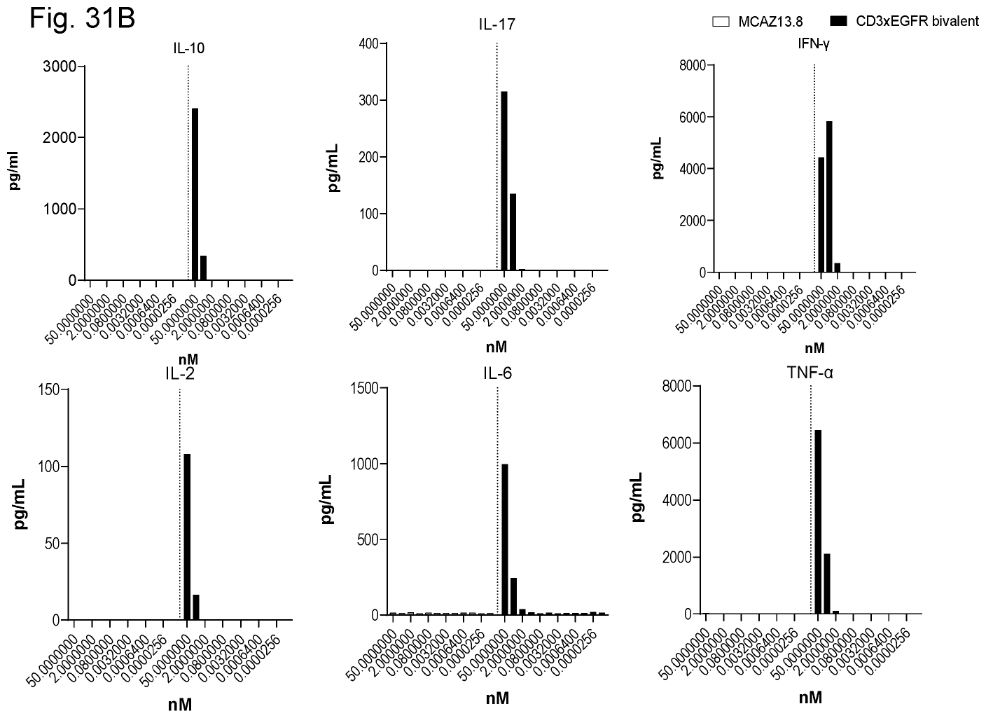

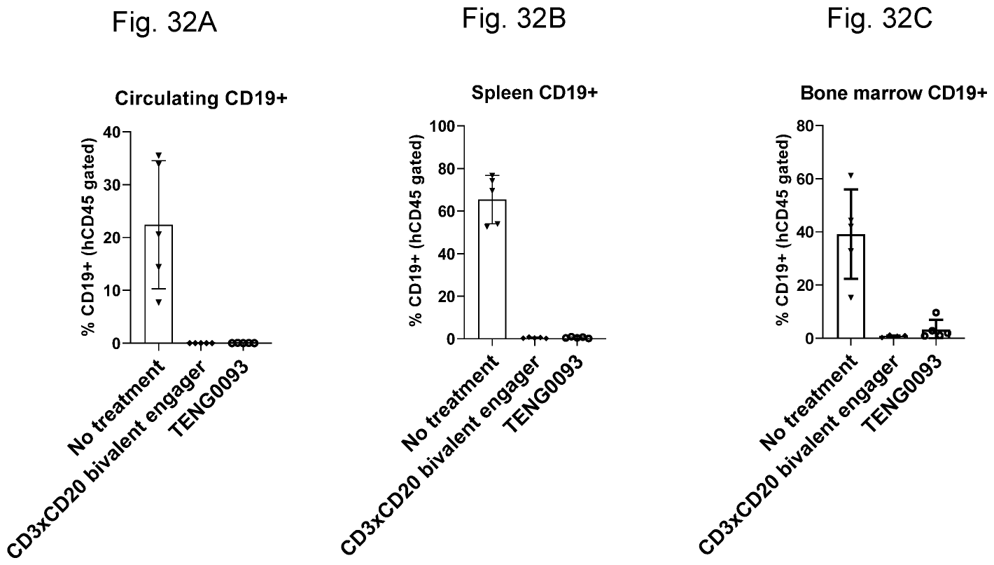

To determine the efficacy of B cell killing and T cell activation of the binding proteins, B cell killing was assessed on PBMCs from NHL (DLBCL) donors. The MCAZ 7.1 variant was spiked in the PBMCs at various concentrations and 48 h later, % B cells was assessed by flow cytometry for each patient. Both CD8-specific T cell engagers, MCAZ 7.1 and the MCAZ 7.1 variant, showed similar strong CD20+ B cell killing on PBMCs from non-Hodgkin lymphoma donors and confirmed strong CD8 T cell-biased activation (FIGS. 24A-24F).

Based on the multitude of binding protein formats tested, a binding protein was identified with a novel conformation comprising the TCR binding domain and the T-cell co-stimulatory domain on separate arms located at the hinge region using an IgG Fc. This binding protein advantageously had increased cytolytic activity and reduced incidence of cytokine release both in vitro and in vivo in the absence of tumor target cells (non-specific T cell activation) and during cytolytic activity in the presence of tumor target cells. Additionally, the binding formats dislcosed herein provide reduced CD4 engagement compared to broad CD3 engagers including (i) limiting Treg activation and proliferation and limiting potential suppressive activity on CD8 cytolytic T cells, and (ii) reducing the engagement of other CD4 T cells subsets (Th1, Th2, Th9, TH17) all of which have been shown to be involved in that have been shown to be drivers of cytokine release syndrome events.

The aspects illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the aspects claimed. Thus, it should be understood that although the present description has been specifically disclosed by aspects, optional features, modification and variation of the concepts herein disclosed may be resorted to by those skilled in the art, and that such modifications and variations are considered to be within the scope of these aspects as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes aspects in which exactly one member of the group is present in, employed in, or otherwise relevant to a given product or process. The disclosure includes aspects in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any elements can be removed from the group.

It should it be understood that, in general, where the disclosure, or aspects of the disclosure, is/are referred to as comprising particular elements and/or features, certain aspects of the disclosure or aspects of the disclosure consist, or consist essentially of, such elements and/or features. For purposes of simplicity, those aspects have not been specifically set forth in haec verba herein.

| SEQ ID | | |
|---|---|---|
| NO: | Description | Sequence |
| 1 | MCAZ6.9 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 2 | MCAZ6.9 Heavy Chain | QVQLQQPGAELVKPGASVKMSCKASGYTFTSY |
| | 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKT.E |
| | | VQLVESGGGLVQPGGSLRLSCVASGDVHKINFL |
| | | GWYRQAPGKEREKVAHISIGDQTDYADSAKGR |
| | | FTISRDESKNMVYLQMNSLKPEDTAVYFCRAFS |
| | | RIYPYDYWGQGTLVTVSS.GEGTSTGSGAIPVSL |
| | | RGSGGSGGAEPKSSDKTHTCPPCPAPEFEGGPSV |
| | | FLFPPKPKDTLMISRTPEVTCVVVDVSHEDPEVK |
| | | FNWYVDGVEVHNAKTKPREEQYNSTYRVVSVL |
| | | TVLHQDWLNGKEYKCKVSNKALPASIEKTISKA |
| | | KGQPREPQVCTLPPSREEMTKNQVSLSCAVKGF |
| | | YPSDIAVEWESNGQPENNYKTTPPVLDSDGSFFL |
| | | VSKLTVDKSRWQQGNVFSCSVMHEALHNRFTQ |
| | | KSLSLSPGK |
| 3 | MCAZ6.9 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GSQVQLQESGGGLVQAGGSLRLSCAASGFTFDD |
| | | YAIGWFRQAPGKEREGVSCIRVSDGSTYYADPV |
| | | KGRFTISSDNAKNTVYLQMNSLKPEDAAVYYC |
| | | AAGSLYTCVQSIVWPARPYYDMDYWGKGTQV |
| | | TVSSAAAYPYDVPDYGS.GEGTSTGSGAIPVSLR |
| | | GSGGSGGAEPKSVDKTHTCPPCPAPEFEGGPSVF |
| | | LFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALPASIEKTISKAK |
| | | GQPREPQVYTLPPCREEMTKNQVSLWCLVKGF |
| | | YPSDIAVEWESNGQPENNYKTTPPVLDSDGSFFL |
| | | YSKLTVDKSRWQQGNVFSCSVMHEALHNHYT |
| | | QKSLSLSPGK |
| 1 | MCAZ6.10 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 2 | MCAZ6.10 Heavy Chain | QVQLQQPGAELVKPGASVKMSCKASGYTFTSY |
| | 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSS.GEGTSTGSGAIPVSLR |
| | | GSGGSGGAEPKSSDKTHTCPPCPAPEFEGGPSVF |
| | | LFPPKPKDTLMISRTPEVTCVVVDVSHEDPEVKF |
| | | NWYVDGVEVHNAKTKPREEQYNSTYRVVSVLT |
| | | VLHQDWLNGKEYKCKVSNKALPASIEKTISKAK |
| | | GQPREPQVCTLPPSREEMTKNQVSLSCAVKGFY |
| | | PSDIAVEWESNGQPENNYKTTPPVLDSDGSFFLV |
| | | SKLTVDKSRWQQGNVFSCSVMHEALHNRFTQK |
| | | SLSLSPGK |
| 4 | MCAZ6.10 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GS.EVQLVESGGGLVQPGGSLRLSCAASGFTFSS |
| | | HWMTWFRQAPGKGLEWVAHIKEDGSEKYYED |
| | | SVEGRFTVSRDNAKNSVYLQMNSLRAEDTAVY |
| | | YCARGGDGYSDSHFGVDVWGQGTTVTVSS.GE |
| | | GTSTGSGAIPVSLRGSGGSGGAEPKSVDKTHTCP |
| | | PCPAPEFEGGPSVFLFPPKPKDTLMISRTPEVTCV |
| | | VVDVSHEDPEVKFNWYVDGVEVHNAKTKPREE |
| | | QYNSTYRVVSVLTVLHQDWLNGKEYKCKVSN |
| | | KALPASIEKTISKAKGQPREPQVYTLPPCREEMT |
| | | KNQVSLWCLVKGFYPSDIAVEWESNGQPENNY |
| | | KTTPPVLDSDGSFFLYSKLTVDKSRWQQGNVFS |
| | | CSVMHEALHNHYTQKSLSLSPGK |
| 1 | MCAZ 88 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 5 | MCAZ 88 Heavy Chains | EVQLVESGGGLVQPGGSLRLSCVASGDVHKINF |
| | | LGWYRQAPGKEREKVAHISIGDQTDYADSAKG |
| | | RFTISRDESKNMVYLQMNSLKPEDTAVYFCRAF |
| | | SRIYPYDYWGQGTLVTVSSGGGGSGGGSGGGS |
| | | QVQLQQPGAELVKPGASVKMSCKASGYTFTSY |
| | | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTHT |
| | | CPPCPAPELLGGPSVFLFPPKPKDTLMISRTPEVT |
| | | CVVVDVSHEDPEVKFNWYVDGVEVHNAKTKP |
| | | REEQYASTYRVVSVLTVLHQDWLNGKEYKCKV |
| | | SNKALPAPIEKTISKAKGQPREPQVYTLPPSRDE |
| | | LTKNQVSLTCLVKGFYPSDIAVEWESNGQPENN |
| | | YKTTPPVLDSDGSFFLYSKLTVDKSRWQQGNVF |
| | | SCSVMHEALHNHYTQKSLSLSPGK |
| 1 | MCAZ 89 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 6 | MCAZ 89 Heavy Chains | QVQLQESGGGLVQAGGSLRLSCAASGFTEDDY |
| | | AIGWFRQAPGKEREGVSCIRVSDGSTYYADPVK |
| | | GRFTISSDNAKNTVYLQMNSLKPEDAAVYYCA |
| | | AGSLYTCVQSIVWPARPYYDMDYWGKGTQVT |
| | | VSSAAAYPYDVPDYGSGGGGSGGGSGGGSQVQ |
| | | LQQPGAELVKPGASVKMSCKASGYTFTSYNMH |
| | | WVKQTPGRGLEWIGAIYPGNGDTSYNQKFKGK |
| | | ATLTADKSSSTAYMQLSSLTSEDSAVYYCARST |
| | | YYGGDWYFNVWGAGTTVTVSAASTKGPSVFPL |
| | | APSSKSTSGGTAALGCLVKDYFPEPVTVSWNSG |
| | | ALTSGVHTFPAVLQSSGLYSLSSVVTVPSSSLGT |
| | | QTYICNVNHKPSNTKVDKKVEPKSCDKTHTCPP |
| | | CPAPELLGGPSVFLFPPKPKDTLMISRTPEVTCV |
| | | VVDVSHEDPEVKFNWYVDGVEVHNAKTKPREE |
| | | QYASTYRVVSVLTVLHQDWLNGKEYKCKVSN |
| | | KALPAPIEKTISKAKGQPREPQVYTLPPSRDELT |
| | | KNQVSLTCLVKGFYPSDIAVEWESNGQPENNYK |
| | | TTPPVLDSDGSFFLYSKLTVDKSRWQQGNVFSC |
| | | SVMHEALHNHYTQKSLSLSPGK |
| 1 | MCAZ 7.5 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 7 | MCAZ 7.5 Heavy Chain | EVQLVESGGGLVQPGGSLRLSCVASGDVHKINF |
| | 1 | LGWYRQAPGKEREKVAHISIGDQTDYADSAKG |
| | | RFTISRDESKNMVYLQMNSLKPEDTAVYFCRAF |
| | | SRIYPYDYWGQGTLVTVSSGEGTSTGSGGSGGS |
| | | GGAEPKSSDKTHTCPPCPAPEFEGGPSVFLFPPK |
| | | PKDTLMISRTPEVTCVVVDVSHEDPEVKFNWYV |
| | | DGVEVHNAKTKPREEQYNSTYRVVSVLTVLHQ |
| | | DWLNGKEYKCKVSNKALPASIEKTISKAKGQPR |
| | | EPQVCTLPPSREEMTKNQVSLSCAVKGFYPSDIA |
| | | VEWESNGQPENNYKTTPPVLDSDGSFFLVSKLT |
| | | VDKSRWQQGNVFSCSVMHEALHNRFTQKSLSL |
| | | SPGK |
| 8 | MCAZ 7.5 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GSGEGTSTGSGGSGGSGGAEPKSVDKTHTCPPC |
| | | PAPEFEGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNK |
| | | ALPASIEKTISKAKGQPREPQVYTLPPCREEMTK |
| | | NQVSLWCLVKGFYPSDIAVEWESNGQPENNYK |
| | | TTPPVLDSDGSFFLYSKLTVDKSRWQQGNVFSC |
| | | SVMHEALHNHYTQKSLSLSPGK |
| 1 | MCAZ 7.1 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 9 | MCAZ 7.1 Heavy Chain | QVQLQQPGAELVKPGASVKMSCKASGYTFTSY |
| | 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AEPKSSDKTHTCPPCPAPEFEGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPASIEKTISKAKGQPRE |
| | | PQVCTLPPSREEMTKNQVSLSCAVKGFYPSDIA |
| | | VEWESNGQPENNYKTTPPVLDSDGSFFLVSKLT |
| | | VDKSRWQQGNVFSCSVMHEALHNRFTQKSLSL |
| | | SPGK |
| 10 | MCAZ 7.1 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GSQVQLQESGGGLVQAGGSLRLSCAASGFTFDD |
| | | YAIGWFRQAPGKEREGVSCIRVSDGSTYYADPV |
| | | KGRFTISSDNAKNTVYLQMNSLKPEDAAVYYC |
| | | AAGSLYTCVQSIVWPARPYYDMDYWGKGTQV |
| | | TVSSAAAYPYDVPDYGSGEGTSTGSGGSGGSGG |
| | | AEPKSVDKTHTCPPCPAPEFEGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPASIEKTISKAKGQPRE |
| | | PQVYTLPPCREEMTKNQVSLWCLVKGFYPSDIA |
| | | VEWESNGQPENNYKTTPPVLDSDGSFFLYSKLT |
| | | VDKSRWQQGNVFSCSVMHEALHNHYTQKSLSL |
| | | SPGK |
| 1 | MCAZ 10.3 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 11 | MCAZ 10.3 Heavy | QVQLQESGGGLVQAGGSLRLSCAASGFTEDDY |
| | Chain 1 | AIGWFRQAPGKEREGVSCIRVSDGSTYYADPVK |
| | | GRFTISSDNAKNTVYLQMNSLKPEDAAVYYCA |
| | | AGSLYTCVQSIVWPARPYYDMDYWGKGTQVT |
| | | VSSEVQLVESGGGLVQPGGSLRLSCVASGDVHK |
| | | INFLGWYRQAPGKEREKVAHISIGDQTDYADSA |
| | | KGRFTISRDESKNMVYLQMNSLKPEDTAVYFCR |
| | | AFSRIYPYDYWGQGTLVTVSSGEPKSSDKTHTC |
| | | PPCPAPEFEGGPSVFLFPPKPKDTLMISRTPEVTC |
| | | VVVDVSHEDPEVKFNWYVDGVEVHNAKTKPR |
| | | EEQYNSTYRVVSVLTVLHQDWLNGKEYKCKVS |
| | | NKALPASIEKTISKAKGQPREPQVCTLPPSREEM |
| | | TKNQVSLSCAVKGFYPSDIAVEWESNGQPENNY |
| | | KTTPPVLDSDGSFFLVSKLTVDKSRWQQGNVFS |
| | | CSVMHEALHNRFTQKSLSLSPGK |
| 12 | MCAZ 10.3 Heavy | QVQLQQPGAELVKPGASVKMSCKASGYTFTSY |
| | Chain 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKGEP |
| | | KSVDKTHTCPPCPAPEFEGGPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPASIEKTISKAKGQPREPQV |
| | | YTLPPCREEMTKNQVSLWCLVKGFYPSDIAVE |
| | | WESNGQPENNYKTTPPVLDSDGSFFLYSKLTVD |
| | | KSRWQQGNVFSCSVMHEALHNHYTQKSLSLSP |
| | | GK |
| 1 | MCAZ 7.7 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 13 | MCAZ 7.7 Heavy Chain | QVQLQQPGAELVKPGASVKMSCKASGYTFTSY |
| | 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GGGSGGGGSEVQLVESGGGLVQPGGSLRLSCV |
| | | ASGDVHKINFLGWYRQAPGKEREKVAHISIGDQ |
| | | TDYADSAKGRFTISRDESKNMVYLQMNSLKPE |
| | | DTAVYFCRAFSRIYPYDYWGQGTLVTVSSGEGT |
| | | STGSGAIPVSLRGSGGSGGAEPKSSDKTHTCPPC |
| | | PAPEFEGGPSVFLFPPKPKDTLMISRTPEVTCVV |
| | | VDVSHEDPEVKFNWYVDGVEVHNAKTKPREEQ |
| | | YNSTYRVVSVLTVLHQDWLNGKEYKCKVSNK |
| | | ALPASIEKTISKAKGQPREPQVCTLPPSREEMTK |
| | | NQVSLSCAVKGFYPSDIAVEWESNGQPENNYKT |
| | | TPPVLDSDGSFFLVSKLTVDKSRWQQGNVFSCS |
| | | VMHEALHNRFTQKSLSLSPGK |
| 14 | MCAZ 7.7 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GSQVQLQESGGGLVQAGGSLRLSCAASGFTFDD |
| | | YAIGWFRQAPGKEREGVSCIRVSDGSTYYADPV |
| | | KGRFTISSDNAKNTVYLQMNSLKPEDAAVYYC |
| | | AAGSLYTCVQSIVWPARPYYDMDYWGKGTQV |
| | | TVSSGEGTSTGSGAIPVSLRGSGGSGGAEPKSVD |
| | | KTHTCPPCPAPEFEGGPSVFLFPPKPKDTLMISRT |
| | | PEVTCVVVDVSHEDPEVKFNWYVDGVEVHNA |
| | | KTKPREEQYNSTYRVVSVLTVLHQDWLNGKEY |
| | | KCKVSNKALPASIEKTISKAKGQPREPQVYTLPP |
| | | CREEMTKNQVSLWCLVKGFYPSDIAVEWESNG |
| | | QPENNYKTTPPVLDSDGSFFLYSKLTVDKSRWQ |
| | | QGNVFSCSVMHEALHNHYTQKSLSLSPGK |
| 1 | MCAZ 10.1 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 15 | MCAZ 10.1 Heavy | QVQLQQPGAELVKPGASVKMSCKASGYTFTSY |
| | Chain 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEPKSVDKTHTCPPCP |
| | | APEFEGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | PASIEKTISKAKGQPREPQVYTLPPCREEMTKNQ |
| | | VSLWCLVKGFYPSDIAVEWESNGQPENNYKTTP |
| | | PVLDSDGSFFLYSKLTVDKSRWQQGNVFSCSV |
| | | MHEALHNHYTQKSLSLSPGK |
| 16 | MCAZ 10.1 Heavy | QVQLQQPGAELVKPGASVKMSCKASGYTFTSY |
| | Chain 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKQV |
| | | QLQESGGGLVQAGGSLRLSCAASGFTFDDYAIG |
| | | WFRQAPGKEREGVSCIRVSDGSTYYADPVKGRF |
| | | TISSDNAKNTVYLQMNSLKPEDAAVYYCAAGS |
| | | LYTCVQSIVWPARPYYDMDYWGKGTQVTVSSG |
| | | EPKSVDKTHTCPPCPAPEFEGGPSVFLFPPKPKD |
| | | TLMISRTPEVTCVVVDVSHEDPEVKFNWYVDG |
| | | VEVHNAKTKPREEQYNSTYRVVSVLTVLHQDW |
| | | LNGKEYKCKVSNKALPASIEKTISKAKGQPREP |
| | | QVYTLPPCREEMTKNQVSLWCLVKGFYPSDIAV |
| | | EWESNGQPENNYKTTPPVLDSDGSFFLYSKLTV |
| | | DKSRWQQGNVFSCSVMHEALHNHYTQKSLSLS |
| | | PGK |
| 17 | MCAZ 8.71 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 18 | MCAZ 8.71 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGAIPVSLR |
| | | GSGGSGGAEPKSSDKTHTCPPCPAPELLGGPSVF |
| | | LFPPKPKDTLMISRTPEVTCVVVDVSHEDPEVKF |
| | | NWYVDGVEVHNAKTKPREEQYASTYRVVSVLT |
| | | VLHQDWLNGKEYKCKVSNKALPAPIEKTISKAK |
| | | GQPREPQVYTLPPSRDELTKNQVSLTCLVKGFY |
| | | PSDIAVEWESNGQPENNYKTTPPVLDSDGSFFLY |
| | | SKLTVDKSRWQQGNVFSCSVMHEALHNHYTQ |
| | | KSLSLSPGK |
| 1 | MCAZ 8.81 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 19 | MCAZ 8.81 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AEPKSCDKTHTCPPCPAPELLGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPAPIEKTISKAKGQPRE |
| | | PQVYTLPPSRDELTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSLS |
| | | PGKGGSGGSQVQLQESGGGLVQAGGSLRLSCA |
| | | ASGFTFDDYAIGWFRQAPGKEREGVSCIRVSDG |
| | | STYYADPVKGRFTISSDNAKNTVYLQMNSLKPE |
| | | DAAVYYCAAGSLYTCVQSIVWPARPYYDMDY |
| | | WGKGTQVTVSS |
| 1 | MCAZ 8.69 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 20 | MCAZ 8.69 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTG |
| | | GSQVQLQESGGGLVQAGGSLRLSCAASGFTFDD |
| | | YAIGWFRQAPGKEREGVSCIRVSDGSTYYADPV |
| | | KGRFTISSDNAKNTVYLQMNSLKPEDAAVYYC |
| | | AAGSLYTCVQSIVWPARPYYDMDYWGKGTQV |
| | | TVSSAAAYPYDVPDYGSGEGTSTGSGAIPVSLR |
| | | GSGGSGGAEPKSVDKTHTCPPCPAPELLGGPSVF |
| | | LFPPKPKDTLMISRTPEVTCVVVDVSHEDPEVKF |
| | | NWYVDGVEVHNAKTKPREEQYASTYRVVSVLT |
| | | VLHQDWLNGKEYKCKVSNKALPAPIEKTISKAK |
| | | GQPREPQVYTLPPSRDELTKNQVSLTCLVKGFY |
| | | PSDIAVEWESNGQPENNYKTTPPVLDSDGSFFLY |
| | | SKLTVDKSRWQQGNVFSCSVMHEALHNHYTQ |
| | | KSLSLSPGK |
| 1 | MCAZ 8.70 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 21 | MCAZ 8.70 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | TYPYDYWGQGTLVTVSSGEGTSTGSGAIPVSLR |
| | | GSGGSGGAEPKSSDKTHTCPPCPAPELLGGPSVF |
| | | LFPPKPKDTLMISRTPEVTCVVVDVSHEDPEVKF |
| | | NWYVDGVEVHNAKTKPREEQYASTYRVVSVLT |
| | | VLHQDWLNGKEYKCKVSNKALPAPIEKTISKAK |
| | | GQPREPQVYTLPPSRDELTKNQVSLTCLVKGFY |
| | | PSDIAVEWESNGQPENNYKTTPPVLDSDGSFFLY |
| | | SKLTVDKSRWQQGNVFSCSVMHEALHNHYTQ |
| | | KSLSLSPGK |
| 1 | TENG0093 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGEC |
| 22 | TENG0093 Heavy Chain | QVQLQQPGAELVKPGASVKMSCKASGYTFTSY |
| | 1 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKRVEPKSCDKTGG |
| | | SEVQLVESGGGLVQPGGSLRLSCVASGDVHKIN |
| | | FLGWYRQAPGKEREKVAHISIGDQTDYADSAK |
| | | GRFTISRDESKNMVYLQMNSLKPEDTAVYFCRA |
| | | FSRIYPYDYWGQGTLVTVSSGGGGSTHTCPPCP |
| | | APEFEGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | PASIEKTISKAKGQPREPQVCTLPPSREEMTKNQ |
| | | VSLSCAVKGFYPSDIAVEWESNGQPENNYKTTP |
| | | PVLDSDGSFFLVSKLTVDKSRWQQGNVFSCSV |
| | | MHEALHNRFTQKSLSLSPGK |
| 23 | TENG0093 Heavy Chain | QVQLQQPGAELVKPGASVKMSCKASGYTFTSY |
| | 2 | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKRVEPKSCDKTGG |
| | | SQVQLQESGGGLVQAGGSLRLSCAASGFTEDDY |
| | | AIGWFRQAPGKEREGVSCIRVSDGSTYYADPVK |
| | | GRFTISSDNAKNTVYLQMNSLKPEDAAVYYCA |
| | | AGSLYTCVQSIVWPARPYYDMDYWGKGTQVT |
| | | VSSGGGGSTHTCPPCPAPEFEGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPASIEKTISKAKGQPRE |
| | | PQVYTLPPCREEMTKNQVSLWCLVKGFYPSDIA |
| | | VEWESNGQPENNYKTTPPVLDSDGSFFLYSKLT |
| | | VDKSRWQQGNVFSCSVMHEALHNHYTQKSLSL |
| | | SPGK |
| 25 | Polypeptide 1 | VQLVQSGAEVKKPGSSVKVSCKASGYAFSYSWI |
| | CD3xCD20 bivalent | NWVRQAPGQGLEWMGRIFPGDGDTDYNGKFK |
| | engager | GRVTITADKSTSTAYMELSSLRSEDTAVYYCAR |
| | | NVFDGYWLVYWGQGTLVTVSSASTKGPSVFPL |
| | | APSSKSTSGGTAALGCLVEDYFPEPVTVSWNSG |
| | | ALTSGVHTFPAVLQSSGLYSLSSVVTVPSSSLGT |
| | | QTYICNVNHKPSNTKVDEKVEPKSCDKTHTCPP |
| | | CPAPEAAGGPSVFLFPPKPKDTLMISRTPEVTCV |
| | | VVDVSHEDPEVKFNWYVDGVEVHNAKTKPREE |
| | | QYNSTYRVVSVLTVLHQDWLNGKEYKCKVSN |
| | | KALGAPIEKTISKAKGQPREPQVCTLPPSRDELT |
| | | KNQVSLSCAVKGFYPSDIAVEWESNGQPENNY |
| | | KTTPPVLDSDGSFFLVSKLTVDKSRWQQGNVFS |
| | | CSVMHEALHNHYTQKSLLSPGK |
| 26 | Polypeptide 2 | VQLVQSGAEVKKPGSSVKVSCKASGYAFSYSWI |
| | CD3xCD20 bivalent | NWVRQAPGQGLEWMGRIFPGDGDTDYNGKFK |
| | engager | GRVTITADKSTSTAYMELSSLRSEDTAVYYCAR |
| | | NVFDGYWLVYWGQGTLVTVSSASTKGPSVFPL |
| | | APSSKSTSGGTAALGCLVEDYFPEPVTVSWNSG |
| | | ALTSGVHTFPAVLQSSGLYSLSSVVTVPSSSLGT |
| | | QTYICNVNHKPSNTKVDEKVEPKSCDGGGGSG |
| | | GGGSQAVVTQEPSLTVSPGGTVTLTCGSSTGAV |
| | | TTSNYANWVQEKPGQAFRGLIGGTNKRAPGTP |
| | | ARFSGSLLGGKAALTLSGAQPEDEAEYYCALW |
| | | YSNLWVFGGGTKLTVLSSASTKGPSVFPLAPSS |
| | | KSTSGGTAALGCLVKDYFPEPVTVSWNSGALTS |
| | | GVHTFPAVLQSSGLYSLSSVVTVPSSSLGTQTYI |
| | | CNVNHKPSNTKVDKKVEPKSCDKTHTCPPCPAP |
| | | EAAGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALG |
| | | APIEKTISKAKGQPREPQVYTLPPCRDELTKNQV |
| | | SLWCLVKGFYPSDIAVEWESNGQPENNYKTTPP |
| | | VLDSDGSFFLYSKLTVDKSRWQQGNVFSCSVM |
| | | HEALHNHYTQKSLSLSPGK |
| 27 | Polypeptide 3 | DIVMTQTPLSLPVTPGEPASISCRSSKSLLHSNGI |
| | CD3xCD20 bivalent | TYLYWYLQKPGQSPQLLIYQMSNLVSGVPDRFS |
| | engager | GSGSGTDFTLKISRVEAEDVGVYYCAQNLELPY |
| | | TFGGGTKVEIKRTVAAPSVFIFPPSDRKLKSGTA |
| | | SVVCLLNNFYPREAKVQWKVDNALQSGNSQES |
| | | VTEQDSKDSTYSLSSTLTLSKADYEKHKVYACE |
| | | VTHQGLSSPVTKSFNRGEC |
| 28 | Polypeptide 4 | EVQLLESGGGLVQPGGSLRLSCAASGFTFSTYA |
| | CD3xCD20 bivalent | MNWVRQAPGKGLEWVSRIRSKYNNYATYYAD |
| | engager | SVKGRFTISRDDSKNTLYLQMNSLRAEDTAVYY |
| | | CVRHGNFGNSYVSWFAYWGQGTLVTVSSASVA |
| | | APSVFIFPPSDEQLKSGTASVVCLLNNFYPREAK |
| | | VQWKVDNALQSGNSQESVTEQDSKDSTYSLSST |
| | | LTLSKADYEKHKVYACEVTHQGLSSPVTKSFNR |
| | | GEC |
| 29 | MCAZ 11.1 Light | QIVLSQSPAILSASPGEKVTMTCRASSSVSYIHW |
| | Chains | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 30 | MCAZ 11.1 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AEPKSSDKTHTCPPCPAPELLGGPSVFLFPPKPK |
| | | DTLMISRTPEVTCVVVDVSHEDPEVKFNWYVD |
| | | GVEVHNAKTKPREEQYASTYRVVSVLTVLHQD |
| | | WLNGKEYKCKVSNKALPAPIEKTISKAKGQPRE |
| | | PQVYTLPPSRDELTKNQVSLTCLVKGFYPSDIAV |
| | | EWESNGQPENNYKTTPPVLDSDGSFFLYSKLTV |
| | | DKSRWQQGNVFSCSVMHEALHNHYTQKSLSLS |
| | | PG |
| 31 | MCAZ 11.2 Light | QIVLSQSPAILSASPGEKVTMTCRASSSVSYIHW |
| | Chains | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 32 | MCAZ 11.2 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AERKCCVECPPCPAPPVAGPSVFLFPPKPKDTLM |
| | | ISRTPEVTCVVVDVSHEDPEVQFNWYVDGVEV |
| | | HNAKTKPREEQFNSTFRVVSVLTVVHQDWLNG |
| | | KEYKCKVSNKGLPAPIEKTISKTKGQPREPQVYT |
| | | LPPSREEMTKNQVSLTCLVKGFYPSDISVEWESN |
| | | GQPENNYKTTPPMLDSDGSFFLYSKLTVDKSRW |
| | | QQGNVFSCSVMHEALHNHYTQKSLSLSPG |
| 33 | MCAZ 11.3 Light | QIVLSQSPAILSASPGEKVTMTCRASSSVSYIHW |
| | Chains | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 34 | MCAZ 11.3 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AELKTPLGDTTHTCPRCPEPKSCDTPPPCPRCPEP |
| | | KSCDTPPPCPRCPEPKSCDTPPPCPRCPAPELLGG |
| | | PSVFLFPPKPKDTLMISRTPEVTCVVVDVSHEDP |
| | | EVQFKWYVDGVEVHNAKTKPREEQYNSTFRVV |
| | | SVLTVLHQDWLNGKEYKCKVSNKALPAPIEKTI |
| | | SKTKGQPREPQVYTLPPSREEMTKNQVSLTCLV |
| | | KGFYPSDIAVEWESSGQPENNYNTTPPMLDSDG |
| | | SFFLYSKLTVDKSRWQQGNIFSCSVMHEALHNR |
| | | FTQKSLSLSPG |
| 35 | MCAZ 11.4 Light | QIVLSQSPAILSASPGEKVTMTCRASSSVSYIHW |
| | Chains | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 36 | MCAZ 11.4 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AESKYGPPCPSCPAPEFLGGPSVFLFPPKPKDTL |
| | | MISRTPEVTCVVVDVSQEDPEVQFNWYVDGVE |
| | | VHNAKTKPREEQFNSTYRVVSVLTVLHQDWLN |
| | | GKEYKCKVSNKGLPSSIEKTISKAKGQPREPQVY |
| | | TLPPSQEEMTKNQVSLTCLVKGFYPSDIAVEWE |
| | | SNGQPENNYKTTPPVLDSDGSFFLYSRLTVDKS |
| | | RWQEGNVFSCSVMHEALHNHYTQKSLSLSLG |
| 37 | MCAZ 11.5 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 38 | MCAZ 11.5 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | AEPKSCDKTHTCPPCPAPELLGGPSVFLFPPKPK |
| | | DTLMISRTPEVTCVVVDVSHEDPEVKFNWYVD |
| | | GVEVHNAKTKPREEQYNSTYRVVSVLTVLHQD |
| | | WLNGKEYKCKVSNKALPAPIEKTISKAKGQPRR |
| | | PQVYTLPPSRDELTKNQVSLTCLVKGFYPSDIAV |
| | | EWESNGQPENNYKTTPPVLDSDGSFFLYSKLTV |
| | | DKSRWQQGNVFSCSVMHGALHNHYTQKYLSLS |
| | | PG |
| 39 | MCAZ 11.6 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 40 | MCAZ 11.6 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGESPKAQASSVPTAQP |
| | | QAEGSLAKATTAPATTRNTGRGGEEKKKEKEK |
| | | EEQEERETKTPECPSHTQPLGVYLLTPAVQDLW |
| | | LRDKATFTCFVVGSDLKDAHLTWEVAGKVPTG |
| | | GVEEGLLERHSNGSQSQHSRLTLPRSLWNAGTS |
| | | VTCTLNHPSLPPQRLMALREPAAQAPVKLSLNL |
| | | LASSDPPEAASWLLCEVSGFSPPNILLMWLEDQ |
| | | REVNTSGFAPARPPPQPRSTTFWAWSVLRVPAP |
| | | PSPQPATYTCVVSHEDSRTLLNASRSLEVSYVTD |
| | | HGPM |
| 41 | MCAZ 11.7 Light Chains | QIVLSQSPAILSASPGEKVTMTCRASSSVSYIHW |
| | | FQQKPGSSPKPWIYATSNLASGVPVRFSGSGSGT |
| | | SYSLTISRVEAEDAATYYCQQWTSNPPTFGGGT |
| | | KLEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGL |
| | | SSPVTKSFNRGECGGGGSGGGGGSGGGGSQVQ |
| | | LQESGGGLVQAGGSLRLSCAASGFTFDDYAIGW |
| | | FRQAPGKEREGVSCIRVSDGSTYYADPVKGRFTI |
| | | SSDNAKNTVYLQMNSLKPEDAAVYYCAAGSLY |
| | | TCVQSIVWPARPYYDMDYWGKGTQVTVSS |
| 42 | MCAZ 11.7 Heavy | QVQLQQPGAELVKPGASVKMSCKASGYTFTSY |
| | Chains | NMHWVKQTPGRGLEWIGAIYPGNGDTSYNQKF |
| | | KGKATLTADKSSSTAYMQLSSLTSEDSAVYYCA |
| | | RSTYYGGDWYFNVWGAGTTVTVSAASTKGPSV |
| | | FPLAPSSKSTSGGTAALGCLVKDYFPEPVTVSW |
| | | NSGALTSGVHTFPAVLQSSGLYSLSSVVTVPSSS |
| | | LGTQTYICNVNHKPSNTKVDKKVEPKSCDKTEV |
| | | QLVESGGGLVQPGGSLRLSCVASGDVHKINFLG |
| | | WYRQAPGKEREKVAHISIGDQTDYADSAKGRF |
| | | TISRDESKNMVYLQMNSLKPEDTAVYFCRAFSR |
| | | IYPYDYWGQGTLVTVSSGEGTSTGSGGSGGSGG |
| | | TPPTPSPSTPPTPSPSCCHPRLSLHRPALEDLLLGS |
| | | EANLTCTLTGLRDASGVTFTWTPSSGKSAVQGP |
| | | PERDLCGCYSVSSVLPGCAEPWNHGKTFTCTAA |
| | | YPESKTPLTATLSKSGNTFRPEVHLLPPPSEELAL |
| | | NELVTLTCLARGFSPKDVLVRWLQGSQELPREK |
| | | YLTWASRQEPSQGTTTFAVTSILRVAAEDWKKG |
| | | DTFSCMVGHEALPLAFTQKTIDRLAGKPTHVNV |
| | | SVVMAEVDGTCY |
| 43 | EGFR TITAN | QVQLKQSGPGLVQPSQSLSITCTVSGFSLTNYGV |
| | (MCAZ13.8): | HWVRQSPGKGLEWLGVIWSGGNTDYNTPFTSR |
| | EGFR HC Cetuximab | LSINKDNSKSQVFFKMNSLQSNDTAIYYCARAL |
| | + linker + linker +TCR | TYYDYEFAYWGQGTLVTVSAASTKGPSVFPLAP |
| | VHH + linker + linker + | SSKSTSGGTAALGCLVKDYFPEPVTVSWNSGAL |
| | Fc domain | TSGVHTFPAVLQSSGLYSLSSVVTVPSSSLGTQT |
| | | YICNVNHKPSNTKVDKKVEPKSCDKTGGSEVQL |
| | | VESGGGLVQPGGSLRLSCVASGDVHKINFLGW |
| | | YRQAPGKEREKVAHISIGDQTDYADSAKGRFTIS |
| | | RDESKNMVYLQMNSLKPEDTAVYFCRAFSRIYP |
| | | YDYWGQGTLVTVSSGGGGSGGGGSGGSGEPKS |
| | | SDKTHTCPPCPAPEFEGGPSVFLFPPKPKDTLMIS |
| | | RTPEVTCVVVDVSHEDPEVKFNWYVDGVEVHN |
| | | AKTKPREEQYNSTYRVVSVLTVLHQDWLNGKE |
| | | YKCKVSNKALPASIEKTISKAKGQPREPQVCTLP |
| | | PSREEMTKNQVSLSCAVKGFYPSDIAVEWESNG |
| | | QPENNYKTTPPVLDSDGSFFLVSKLTVDKSRWQ |
| | | QGNVFSCSVMHEALHNRFTQKSLSLSPGK |
| 44 | EGFR TITAN | EVQLVESGGGLVQAGGSLRLSCAASGRTFSSYA |
| | (MCAZ13.8): | MGWFRQAPGKEREFVVAINWSSGSTYYADSVK |
| | EGFR VHH 9G8+ | GRFTISRDNAKNTMYLQMNSLKPEDTAVYYCA |
| | Linker+ | AGYQINSGNYNFKDYEYDYWGQGTQVTVSSTG |
| | CD8 VHH | GSQVQLQESGGGLVQAGGSLRLSCAASGFTFDD |
| | linker + linker + | YAIGWFRQAPGKEREGVSCIRVSDGSTYYADPV |
| | FC domain | KGRFTISSDNAKNTVYLQMNSLKPEDAAVYYC |
| | | AAGSLYTCVQSIVWPARPYYDMDYWGKGTQV |
| | | TVSSGGGGSGGGGSGSEPKSVDKTHTCPPCPAP |
| | | EFEGGPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALPA |
| | | SIEKTISKAKGQPREPQVYTLPPCREEMTKNQVS |
| | | LWCLVKGFYPSDIAVEWESNGQPENNYKTTPPV |
| | | LDSDGSFFLYSKLTVDKSRWQQGNVFSCSVMH |
| | | EALHNHYTQKSLSLSPGK |
| 45 | EGFR TITAN | DILLTQSPVILSVSPGERVSFSCRASQSIGTNIHW |
| | (MCAZ13.8): | YQQRTNGSPRLLIKYASESISGIPSRFSGSGSGTD |
| | EGFR LC Cetuximab | FTLSINSVESEDIADYYCQQNNNWPTTFGAGTK |
| | | LELKRTVAAPSVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLS |
| | | SPVTKSFNRGEC |
| 46 | linker | TGGS |
| 47 | linker | GGGGS |
| 48 | linker | AAAYPYDVPDYGSGEGTSTGSGGSGGSGGA |
| 49 | linker | GEGTSTGSGGSGGSGGA |
| 50 | TCR VHH | EVQLVESGGGLVQPGGSLRLSCVASGDVHKINF |
| | | LGWYRQAPGKEREKVAHISIGDQTDYADSAKG |
| | | RFTISRDESKNMVYLQMNSLKPEDTAVYFCRAF |
| | | SRIYPYDYWGQGTLVTVSS |
| 51 | CD8 VHH | QVQLQESGGGLVQAGGSLRLSCAASGFTEDDY |
| | | AIGWFRQAPGKEREGVSCIRVSDGSTYYADPVK |
| | | GRFTISSDNAKNTVYLQMNSLKPEDAAVYYCA |
| | | AGSLYTCVQSIVWPARPYYDMDYWGKGTQVT |
| | | VSS |
| 52 | Fc domain | APEFEGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | PASIEKTISKAKGQPREPQVYTLPPCREEMTKNQ |
| | | VSLWCLVKGFYPSDIAVEWESNGQPENNYKTTP |
| | | PVLDSDGSFFLYSKLTVDKSRWQQGNVFSCSV |
| | | MHEALHNHYTQKSLSLSPGK |
| 53 | EGFR VHH 9G8 | EVQLVESGGGLVQAGGSLRLSCAASGRTFSSYA |
| | | MGWFRQAPGKEREFVVAINWSSGSTYYADSVK |
| | | GRFTISRDNAKNTMYLQMNSLKPEDTAVYYCA |
| | | AGYQINSGNYNFKDYEYDYWGQGTQVTVSS |
| 54 | EGFR HC Cetuximab | QVQLKQSGPGLVQPSQSLSITCTVSGFSLTNYGV |
| | | HWVRQSPGKGLEWLGVIWSGGNTDYNTPFTSR |
| | | LSINKDNSKSQVFFKMNSLQSNDTAIYYCARAL |
| | | TYYDYEFAYWGQGTLVTVSAASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKKV |
| 55 | linker | EPKSCDK |
| 56 | linker | EPKSSDKTHTCPPCP |

1. A binding protein comprising four polypeptide chains that form two tumor-associated antigen binding sites, a T cell receptor binding site, and a T cell co-stimulatory molecule binding site, wherein the first and second polypeptide chains have a structure represented by the formula: 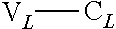
and a third polypeptide chain has a structure represented by the formula: 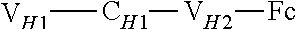
and a fourth polypeptide chain has a structure represented by the formula: 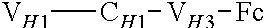
wherein:
V_{L} is an immunoglobulin light chain variable domain that specifically binds a tumor-associated antigen;
V_{H1} is an immunoglobulin heavy chain variable domain that specifically binds a tumor-associated antigen;
C_{L} is an immunoglobulin light chain constant domain that specifically binds a tumor-associated antigen;
C_{H1} is an immunoglobulin CH1 heavy chain constant domain that specifically binds a tumor-associated antigen;
V_{H2} is a heavy chain variable domain that specifically binds a T cell receptor;
V_{H3} is a heavy chain variable domain that specifically binds T cell co-stimulatory molecule; and
Fc is C_{H2} and C_{H3} immunoglobulin heavy chain constant domains.

2. The binding protein of claim 1 further comprising L₁, a linker positioned between C_{H1} and V_{H2} on the third polypeptide chain and L₂, a linker positioned between V_{H2} and the Fc on the third polypeptide chain, wherein L₁ and L₂ are each independently a linker or are absent.

3. The binding protein of claim 1 or claim 2 further comprising L₃, a linker positioned between C_{H1} and V_{H3} on the fourth polypeptide chain and L₄, a linker positioned between V_{H3} and Fc on the fourth polypeptide chain wherein L₃ and L₄ are each independently a linker or are absent.

4. The binding protein of any one of claims 1-3 further comprising H₁, an immunoglobulin hinge region positioned between C_{H1} and V_{H2} on the third polypeptide chain and H₂, an immunoglobulin hinge region positioned between V_{H2} and the Fc on the third polypeptide chain, wherein H₁ and H₂ are each independently an immunoglobulin hinge region or are absent.

5. The binding protein of any one of claims 1-4 further comprising H₃, an immunoglobulin hinge region positioned between C_{H1} and V_{H3} on the fourth polypeptide chain and H₄, an immunoglobulin hinge region positioned between V_{H3} and the Fc on the fourth polypeptide chain, wherein H₃ and H₄ are each independently an immunoglobulin hinge region or are absent.

6. The binding protein of any one of claims 1-5, wherein the first and second polypeptide chains have a structure represented by the formula: 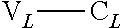
and a third polypeptide chain has a structure represented by the formula: 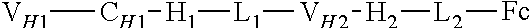
and a fourth polypeptide chain has a structure represented by the formula:

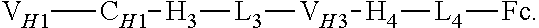

7. The binding protein of any one of claims 1-6, wherein the Fc is from an IgG antibody.

8. The binding protein of any one of claims 1-7, wherein the binding protein activates T cells only when bound to a tumor associated antigen at one or both of the antigen binding sites.

9. The binding protein of any one of claims 1-8, wherein the tumor-associated antigen is CD20.

10. The binding protein of any one of claims 1-9, wherein the T cell costimulatory molecule is CD8 or CD137.

11. A binding protein comprising four polypeptide chains that form two tumor-associated antigen binding sites, a T cell receptor binding site, and a T cell co-stimulatory molecule binding site, wherein two polypeptide chains have a structure represented by the formula: 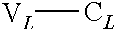
and two polypeptide chains have a structure represented by the formula: 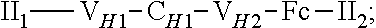
wherein:
V_{L} is an immunoglobulin light chain variable domain that specifically binds a tumor-associated antigen;
V_{H1} is an immunoglobulin heavy chain variable domain that specifically binds a tumor-associated antigen;
C_{L} is an immunoglobulin light chain constant domain that specifically binds a tumor-associated antigen;
C_{H1} is an immunoglobulin CH1 heavy chain constant domain that specifically binds a tumor-associated antigen;
V_{H2} is a heavy chain variable domain that specifically binds a T cell receptor;
Fc is C_{H2} and C_{H3} immunoglobulin heavy chain constant domains;
II₁ and II₂ are each independently a heavy chain variable domain that specifically binds a T cell co-stimulatory molecule or are absent;
wherein at least one of II₁ and II₂ is a heavy chain variable domain that specifically binds a T cell co-stimulatory molecule.

12. A binding protein comprising four polypeptide chains that form two tumor-associated antigen binding sites, a T cell receptor binding site, and a T cell co-stimulatory molecule binding site, wherein two polypeptide chains have a structure represented by the formula: 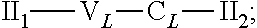
and two polypeptide chains have a structure represented by the formula: 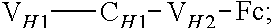
wherein:
V_{L} is an immunoglobulin light chain variable domain that specifically binds a tumor-associated antigen;
V_{H1} is an immunoglobulin heavy chain variable domain that specifically binds a tumor-associated antigen;
C_{L} is an immunoglobulin light chain constant domain that specifically binds a tumor-associated antigen;
C_{H1} is an immunoglobulin CH1 heavy chain constant domain that specifically binds a tumor-associated antigen;
V_{H2} is a heavy chain variable domain that specifically binds a T cell receptor;
Fc is C_{H2} and C_{H3} immunoglobulin heavy chain constant domains;
II₁ and II₂ are each independently a heavy chain variable domain that specifically binds a T cell co-stimulatory molecule or are absent;
wherein at least one of II₁ and II₂ is a heavy chain variable domain that specifically binds a T cell co-stimulatory molecule.

13. A binding protein comprising three polypeptide chains that form two tumor-associated antigen binding sites, a T cell receptor binding site, and a T cell co-stimulatory molecule binding site, wherein the first polypeptide chain has a structure represented by the formula: 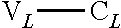
and a second polypeptide chain has a structure represented by the formula: 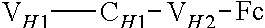
and a third polypeptide chain has a structure represented by the formula: 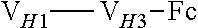
wherein:
V_{L} is an immunoglobulin light chain variable domain that specifically binds a tumor-associated antigen;
V_{H1} is an immunoglobulin heavy chain variable domain that specifically binds a tumor-associated antigen;
C_{L} is an immunoglobulin light chain constant domain that specifically binds a tumor-associated antigen;
C_{H1} is an immunoglobulin CH1 heavy chain constant domain that specifically binds a tumor-associated antigen;
V_{H2} is a heavy chain variable domain that specifically binds a T cell receptor;
V_{H3} is a heavy chain variable domain that specifically binds T cell co-stimulatory molecule; and
Fc is C_{H2} and C_{H3} immunoglobulin heavy chain constant domains.

14. The binding protein of any one of claims 11-13, wherein the binding protein activates T cells only when bound to a tumor associated antigen at one or both of the antigen binding sites.

15. The binding protein of any one of claims 11-14, wherein the tumor-associated antigen is CD20.

16. The binding protein of any one of claims 11-15, wherein the T cell costimulatory molecule is CD8 or CD137.

17. A binding protein comprising four polypeptide chains that form two tumor-associated antigen binding sites, a T cell receptor binding site, and a T cell co-stimulatory molecule binding site, wherein the polypeptide chains comprise
(a) the amino acid of sequences of SEQ ID NO: 1, SEQ ID NO: 2, and SEQ ID NO: 3;
(b) the amino acid of sequences of SEQ ID NO: 1, SEQ ID NO: 2, and SEQ ID NO: 4;
(c) the amino acid of sequences of SEQ ID NO: 1, SEQ ID NO: 9, and SEQ ID NO: 10;
(d) the amino acid of sequences of SEQ ID NO: 1, SEQ ID NO: 11, and SEQ ID NO: 12;
(e) the amino acid of sequences of SEQ ID NO: 1, SEQ ID NO: 13, and SEQ ID NO: 14;
(f) the amino acid of sequences of SEQ ID NO: 1 and SEQ ID NO: 19;
(g) the amino acid of sequences of SEQ ID NO: 1, SEQ ID NO: 22, and SEQ ID NO: 23;
(h) the amino acid of sequences of SEQ ID NO: 29 and SEQ ID NO: 30;
(i) the amino acid of sequences of SEQ ID NO: 31 and SEQ ID NO: 32;
(j) the amino acid of sequences of SEQ ID NO: 33 and SEQ ID NO: 34;
(k) the amino acid of sequences of SEQ ID NO: 35 and SEQ ID NO: 36;
(l) the amino acid of sequences of SEQ ID NO: 37 and SEQ ID NO: 38;
(m) the amino acid of sequences of SEQ ID NO: 39 and SEQ ID NO: 40;
(n) the amino acid of sequences of SEQ ID NO: 41 and SEQ ID NO: 42; or
(o) the amino acid of sequences of SEQ ID NO: 45, and SEQ ID NO: 54.

18. A binding protein comprising three polypeptide chains that form two tumor-associated antigen binding sites, a T cell receptor binding site, and a T cell co-stimulatory molecule binding site, wherein the polypeptide chains comprise the amino acid of sequences of SEQ ID NO: 43, SEQ ID NO: 44, and SEQ ID NO: 45 or (q) the amino acid of sequences of SEQ ID NO: 1, SEQ ID NO: 11 and SEQ ID NO: 12.

19. A pharmaceutical composition comprising the binding protein of any one of claims 1-18 and a pharmaceutically acceptable carrier.

20. An isolated nucleic acid sequence encoding the binding protein of claim 17 or claim 18.

21. A vector comprising the isolated nucleic acid sequence of claim 20.

22. A method of treating cancer, comprising administering to a subject in need thereof an effective amount of the binding protein of any one of claims 1-18.

23. The method of claim 22, wherein the binding protein preferentially activates a subset of T cells in the subject.

24. The method of either claim 22 or 23, wherein the subset of T cells are CD8 T cells.

25. The method of any one of claims 22-24, wherein the CD8 T cells are preferentially activated as compared to CD4 T cells.

26. The method of claim 23, wherein the activation of T cells is determined by measuring the percentage of surface CD25+ T cells.

27. The method of claim 26, wherein the percentage of surface CD25+ T cells that are CD8 T cells is higher than the percentage of surface CD25+ T cells that are CD4 T cells.

28. A method of treating an inflammatory disease, comprising administering to a subject in need thereof an effective amount of the binding protein of any one of claims 1-18.

29. A binding protein according to any one of claims 1-18, or a pharmaceutical composition according to claim 19, for use as a medicament.

30. A binding protein according to any one of claims 1-18, or a pharmaceutical composition according to claim 19, for use in the treatment of cancer.

31. A binding protein according to any one of claims 1-18, or a pharmaceutical composition according to claim 19, for use in the treatment of an inflammatory disease.

32. Use of a binding protein according to any one of claims 1-18, or a pharmaceutical composition according to claim 19, in the manufacture of a medicament for use in the treatment of cancer.

33. Use of a binding protein according to any one of claims 1-18, or a pharmaceutical composition according to claim 19, in the manufacture of a medicament for use in the treatment of an inflammatory disease.
